(19) United States
(12) Patent Application Publication
(10) Pub. No.: US 2026/0114713 A1
(43) Pub. Date: Apr. 30, 2026

(54) ENDOSCOPE

(71) Applicant: FUJIFILM Corporation, Tokyo (JP)

(72) Inventor: Takuro ASAOKA, Kanagawa (JP)

(73) Assignee: FUJIFILM Corporation, Tokyo (JP)

(21) Appl. No.: 19/373,651

(22) Filed: Oct. 29, 2025

(30) Foreign Application Priority Data
Oct. 31, 2024 (JP) 2024-192132

(51) Int. Cl. A61B 1/00; A61B 1/018; A61B 1/233; A61B 17/29

(52) U.S. Cl. CPC A61B 1/00096; A61B 1/00197; A61B 1/018; A61B 1/233; A61B 17/29

(57) ABSTRACT

An endoscope includes: an insertion part; an optical system ; a sensor; a first holding part; a shaft member fixed to the first holding part and extending toward a base end side relative to the first holding part; an actuator that moves the shaft member in an optical axis direction along an optical axis of the optical system; and a first restriction part that restricts a position of the shaft member in a first direction and a second direction perpendicular to the optical axis, a length of the shaft member in the first direction and that in the second direction are shorter than a diameter of a first lens, and a length between a distal end of the first restriction part and a base end of the first restriction part in the optical axis direction is longer than a length of the first lens in the optical axis direction.

### CROSS-REFERENCE TO RELATED APPLICATIONS

This application is based upon and claims the benefit of priority from prior Japanese patent application No. 2024-192132, filed on Oct. 31, 2024, the entire contents of which are incorporated herein by reference.

### BACKGROUND OF THE INVENTION

### 1. Field of the Invention

The technology of the present disclosure relates to an endoscope.

### 2. Description of the Related Art

JP2008-6215A discloses an imaging apparatus for an endoscope, comprising an optical lens and an electrostatic actuator that moves the optical lens in an optical axis direction, in which the electrostatic actuator has a moving object that is connected to the optical lens via a connecting rod, and an electrostatic vibration substrate that is interposed between the optical lens and the moving object and that can move the moving object in the optical axis direction by vibration.

JP2013-75028A discloses an imaging lens unit for an endoscope, including an imaging lens having a movable lens that is movable in an optical axis direction, a lens movement frame that holds the movable lens and moves the movable lens in the optical axis direction, and a camshaft that is provided parallel to an optical axis of the imaging lens and engages with the lens movement frame.

### SUMMARY OF THE INVENTION

The technology of the present disclosure provides an endoscope that can be reduced in diameter.

An aspect of the technology of the present disclosure relates to an endoscope comprising: an insertion part that is inserted into a subject; an optical system that is disposed on a distal end side of the insertion part and includes a first lens; a sensor that captures an optical image of the subject formed by the optical system; a first holding part that holds the first lens; a shaft member that is fixed to the first holding part and extends toward a base end side relative to the first holding part; an actuator that moves the shaft member in an optical axis direction along an optical axis of the optical system; and a first restriction part that restricts a position of the shaft member in directions perpendicular to the optical axis, in which the directions perpendicular to the optical axis include a first direction and a second direction perpendicular to the first direction, a length of the shaft member in the first direction and a length of the shaft member in the second direction are shorter than a diameter of the first lens, and a length between a distal end of the first restriction part and a base end of the first restriction part in the optical axis direction is longer than a length of the first lens in the optical axis direction.

According to the technology of the present disclosure, it is possible to reduce the diameter.

### BRIEF DESCRIPTION OF THE DRAWINGS

FIG. 1 is a schematic view showing an endoscope 1 according to an embodiment of the technology of the present disclosure.

FIG. 2 is an enlarged perspective view of the vicinity of a distal end portion 7 of the endoscope 1.

FIG. 3 is an exploded perspective view of the distal end portion 7 shown in FIG. 2.

FIG. 4 is an enlarged perspective view of a part of the distal end portion 7 seen from a direction different from the direction in FIG. 2.

FIG. 5 is a view of FIG. 4 seen from an up direction U.

FIG. 6 is a view of the distal end portion 7 seen in a distal end direction Fr.

FIG. 7 is a schematic cross-sectional view taken along line A-A shown in FIG. 6.

FIG. 8 is a schematic cross-sectional view taken along line B-B shown in FIG. 7.

FIG. 9 is a schematic view showing a modification example of the disposition of a shaft member 33.

FIG. 10 is a schematic view showing a modification example of the disposition of a sensor 37.

FIG. 11 is a schematic view showing a modification example of a substrate 38.

FIG. 12 is a schematic view showing a modification example of the disposition of a prism 36, the sensor 37, and the substrate 38.

FIG. 13 is a schematic view showing a first modification example of an imaging unit 300 and a housing member 71, and is a view taken along line A-A shown in FIG. 6.

FIG. 14 is a schematic view showing a second modification example of the imaging unit 300 and the housing member 71, and is a view taken along line A-A shown in FIG. 6.

FIG. 15 is a schematic view showing a third modification example of the imaging unit 300 and the housing member 71, and is a view taken along line A-A shown in FIG. 6.

FIG. 16 is a schematic view showing a fourth modification example of the imaging unit 300 and the housing member 71, and is a view taken along line A-A shown in FIG. 6.

FIG. 17 is a schematic view showing a fifth modification example of the imaging unit 300 and the housing member 71, and is a view taken along line A-A shown in FIG. 6.

FIG. 18 is a schematic view showing a sixth modification example of the imaging unit 300 and the housing member 71, and is a view taken along line A-A shown in FIG. 6.

FIG. 19 is a schematic view showing a seventh modification example of the imaging unit 300 and the housing member 71, and is a view taken along line A-A shown in FIG. 6.

FIG. 20 is a schematic view showing an eighth modification example of the imaging unit 300 and the housing member 71, and is a view taken along line A-A shown in FIG. 6.

FIG. 21 is a schematic view showing a first modification example of a second restriction part 322, and is a view taken along line B-B shown in FIG. 7.

FIG. 22 is a schematic view showing a second modification example of the second restriction part 322, and is a view taken along line B-B shown in FIG. 7.

FIG. 23 is a schematic view showing a third modification example of the second restriction part 322, and is a view taken along line B-B shown in FIG. 7.

FIG. 24 is a schematic view showing a fourth modification example of the second restriction part 322, and is a view taken along line B-B shown in FIG. 7.

FIG. 25 is a perspective view showing an external configuration of a housing member 71A that houses the imaging unit 300 of the modification example.

FIG. 26 is a schematic cross-sectional view taken along line C-C shown in FIG. 25.

FIG. 27 is a schematic cross-sectional view taken along line D-D shown in FIG. 26.

FIG. 28 is a schematic view showing a ninth modification example of the imaging unit 300 and the housing member 71, and is a view taken along line A-A shown in FIG. 6.

### DESCRIPTION OF THE PREFERRED EMBODIMENTS

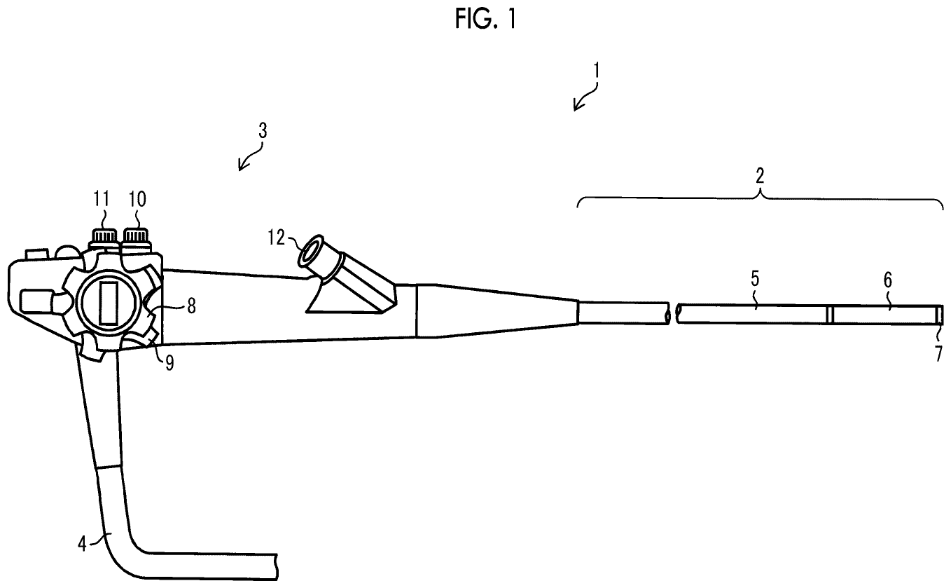

FIG. 1 is a schematic view showing an endoscope 1 according to an embodiment of the technology of the present disclosure. The endoscope 1 in the same drawing comprises an elongated insertion part 2 that is inserted into a subject, an operating part 3 that is connected to a base end of the insertion part 2 and is used for gripping and operating the endoscope 1, and a universal cord 4 that connects the endoscope 1 to system constituent devices such as a light source device and a processor device (not shown). The technology of the present embodiment is not particularly limited, but can be preferably applied to a transnasal endoscope in which a diameter of a distal end portion 7 of the insertion part 2 is 6 mm or less. In addition, the endoscope can be preferably applied to an endoscope in which a difference between the diameter of the distal end portion 7 and a diameter of a forceps port 26 provided in the distal end portion 7 is smaller than 6 mm.

The insertion part 2 includes a soft portion 5, a bendable portion 6, and the distal end portion 7 that are consecutively provided in order from a base end toward a distal end. The soft portion 5 is flexible and can be bent in any direction along an insertion path of the insertion part 2. The operating part 3 includes angle knobs 8 and 9, a treatment tool inlet port 12, an air/water supply button 10, a suction button 11, and the like.

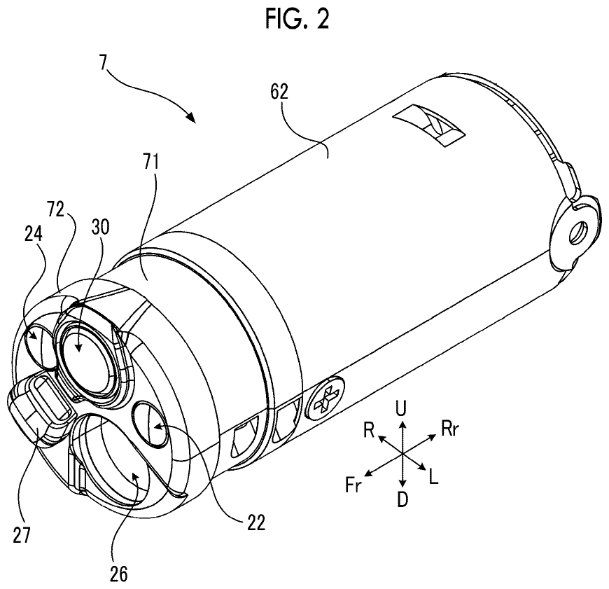

The bendable portion 6 is bent in each of up-down and left-right directions by each operation of the angle knobs 8 and 9. A treatment tool, such as a forceps, is inserted from the treatment tool inlet port 12 and is led out from the forceps port 26 (see FIG. 2) provided in the distal end portion 7. In addition, the distal end portion 7 is provided with an observation window 30 (see FIG. 2) for imaging a part to be observed in a body, and a first illumination window 24 and a second illumination window 22 (see FIG. 2) for irradiating the part to be observed with illumination light.

The insertion part 2 moves along an axial direction thereof and is inserted into the subject, and the angle knobs 8 and 9 of the operating part 3 are rotationally operated to bend the bendable portion 6 of the insertion part 2 in the up-down and left-right directions. As a result, the distal end portion 7 of the insertion part 2 can be directed to a desired direction in the body, and an observation image can be acquired by the observation window 30 provided in the distal end portion 7.

Hereinafter, a direction from a base end to a distal end of the insertion part 2 will be referred to as a distal end direction Fr, and a direction opposite to the distal end direction Fr will be referred to as a base end direction Rr. The distal end direction Fr and the base end direction Rr will be collectively referred to as a longitudinal axis direction of the insertion part 2 or an optical axis direction in which an optical axis AX of an optical system OP described later extends. Two directions perpendicular to each other and perpendicular to the longitudinal axis direction of the insertion part 2 will be referred to as an up-down direction and a left-right direction. One direction in the up-down direction will be referred to as an up direction U, and a side opposite to the up direction U will be referred to as a down direction D. One direction in the left-right direction will be referred to as a right direction R, and a side opposite to the right direction R will be referred to as a left direction L.

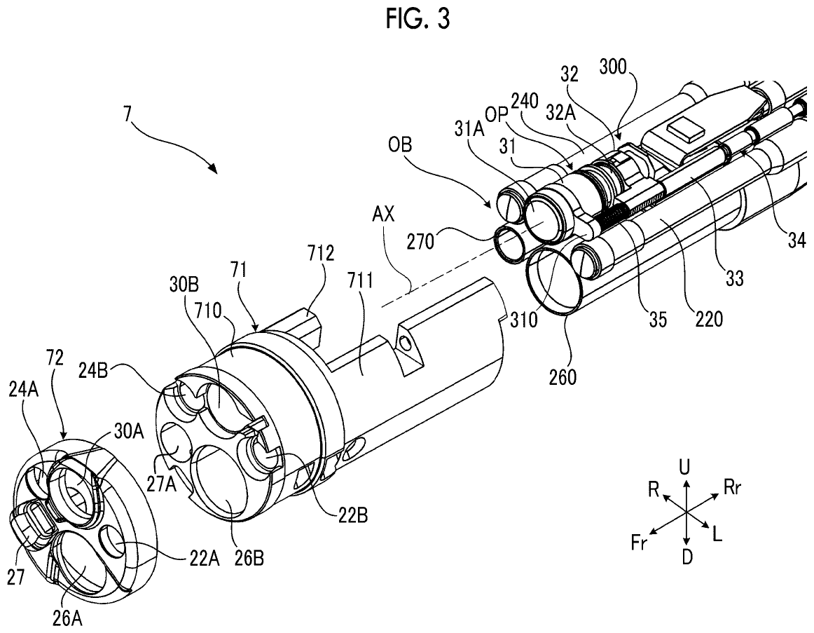

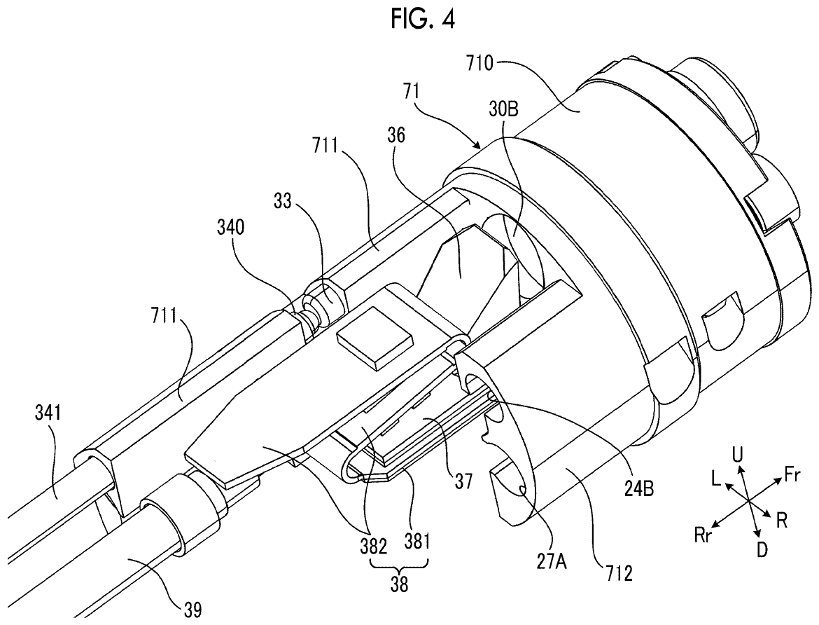

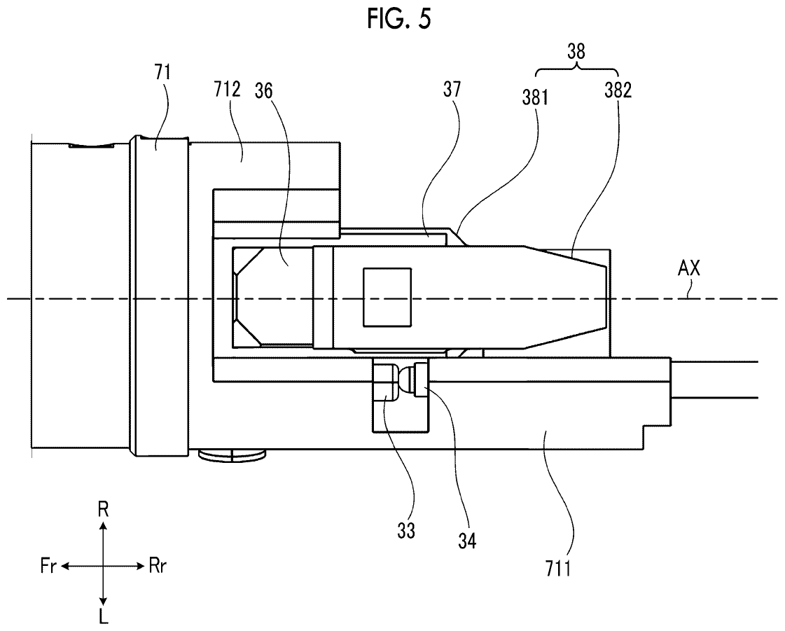

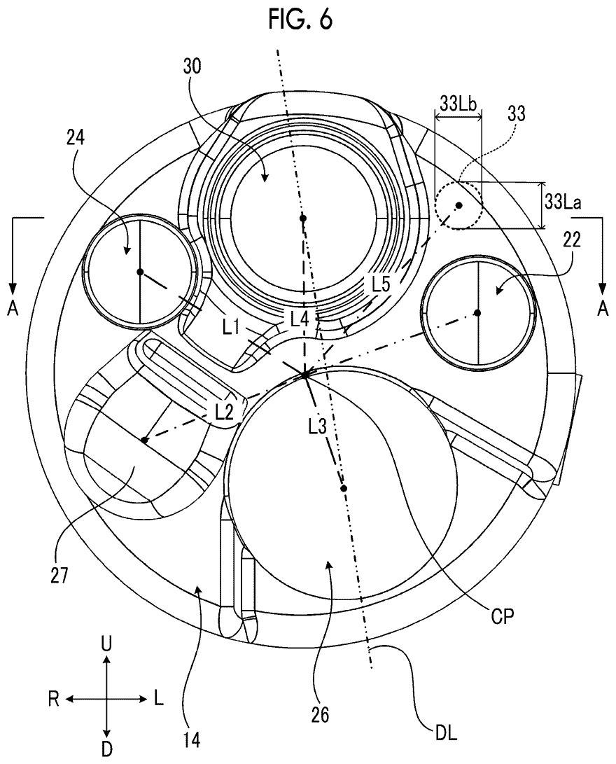

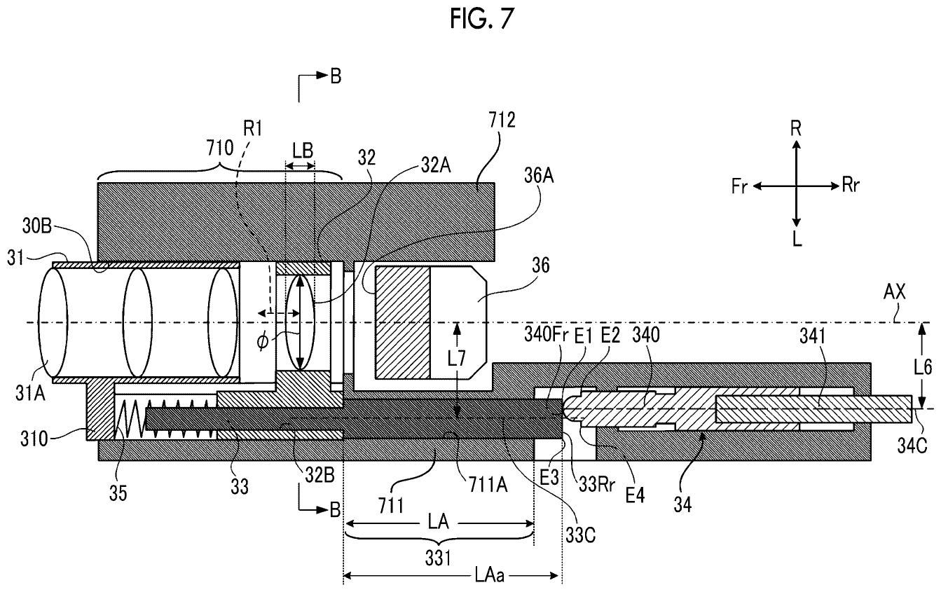

FIG. 2 is an enlarged perspective view of the vicinity of the distal end portion 7 of the endoscope 1. FIG. 3 is an exploded perspective view of the distal end portion 7 shown in FIG. 2. FIG. 4 is an enlarged perspective view of a part of the distal end portion 7 seen from a direction different from the direction in FIG. 2. FIG. 5 is a view of FIG. 4 seen from the up direction U. FIG. 6 is a view of the distal end portion 7 seen from the distal end direction Fr. FIG. 7 is a schematic cross-sectional view taken along line A-A of FIG. 6.

As shown in FIG. 3, the insertion part 2 houses an internal member assembly OB including a plurality of elongated internal members extending from the base end to the distal end. The internal member assembly OB includes an imaging unit 300, a left light guide 220 and a right light guide 240 that are each configured by bundling a plurality of optical fibers, a treatment tool pipe line 260 into which a treatment tool such as forceps or a biopsy needle is inserted, and a fluid pipe line 270 into which liquid or gas flows.

As shown in FIGS. 2 and 3, the distal end portion 7 comprises a housing member 71 that houses and holds the imaging unit 300, the left light guide 220, the right light guide 240, the treatment tool pipe line 260, and the fluid pipe line 270, and a distal end cap 72 fixed to a distal end side of the housing member 71.

The bendable portion 6 has a bending joint portion formed by pivotally connecting a plurality of annular members to each other. As shown in FIG. 2, a connecting member 62 for connecting the housing member 71 of the distal end portion 7 and the bendable portion 6 is provided at the most distal end side in the bending joint portion. The connecting member 62 is fixed to the housing member 71 by screwing or the like, so that the distal end portion 7 and the bendable portion 6 are connected to each other.

As shown in FIG. 3, the housing member 71 comprises a cylindrical body portion 710, an extension portion 711 that extends from a left base end of the body portion 710 in the base end direction Rr, and an extension portion 712 that extends from a right base end of the body portion 710 in the base end direction Rr.

The body portion 710 is provided with a plurality of holes that penetrate in the optical axis direction. The plurality of holes include an insertion hole 24B into which the right light guide 240 is inserted and fixed, an insertion hole 30B into which the imaging unit 300 is inserted and fixed, an insertion hole 22B into which the left light guide 220 is inserted and fixed, an insertion hole 26B into which the treatment tool pipe line 260 is inserted and fixed, and an insertion hole 27A into which the fluid pipe line 270 is inserted and fixed.

The distal end cap 72 is provided with an insertion hole 30A that is disposed to face the insertion hole 30B, an insertion hole 22A that is disposed to face the insertion hole 22B, an insertion hole 24A that is disposed to face the insertion hole 24B, an insertion hole 26A that is disposed to face the insertion hole 26B, and an insertion hole that is disposed to face the insertion hole 27A. An air/water supply nozzle 27 is inserted from the distal end side into the insertion hole disposed to face the insertion hole 27A.

In an assembled state, as shown in FIG. 2, the observation window 30 is defined by a distal end (objective lens 31A) of the imaging unit 300 inserted into the insertion hole 30A. The second illumination window 22 is formed by a distal end of the left light guide 220 inserted into the insertion hole 22A. The first illumination window 24 is formed by a distal end of the right light guide 240 inserted into the insertion hole 24A. The forceps port 26 is formed by a distal end of the treatment tool pipe line 260 inserted into the insertion hole 26A. The observation window 30, the first illumination window 24, the second illumination window 22, the forceps port 26, and the air/water supply nozzle 27 each form a functional element.

The imaging unit 300 includes the optical system OP including a fixed lens holder 31 that holds a fixed lens group including the objective lens 31A, a movable lens holder 32 that holds a movable lens group including at least one movable lens 32A such as a focus lens or a zoom lens, and a prism 36 (see FIG. 4) disposed on a base end side relative to the fixed lens group and the movable lens group. FIG. 3 shows an optical axis AX of the optical system OP.

As shown in FIG. 7, the fixed lens holder 31 is fixed in the insertion hole 30B of the housing member 71. The movable lens holder 32 is provided in the insertion hole 30B so as to be movable in the optical axis direction.

As shown in FIG. 4, the imaging unit 300 comprises a sensor 37 such as a charge-coupled device (CCD) image sensor or a complementary metal-oxide-semiconductor (CMOS) image sensor that captures an optical image of the subject formed by the optical system OP, a substrate 38 on which the sensor 37 is provided, and a cable 39 connected to the substrate 38.

As shown in FIG. 5, the optical system OP is provided such that the optical axis AX thereof is along the longitudinal axis direction of the insertion part 2. The light that passes through the fixed lens group and the movable lens group and is incident on the prism 36 is reflected from a light reflection surface of the prism 36, travels in the down direction D, and is emitted from the light emission surface. A light-receiving surface of the sensor 37 is disposed to face the light emission surface and is disposed along the optical axis AX.

As shown in FIG. 4, the substrate 38 includes a first substrate portion 381 in which the sensor 37 is provided and a second substrate portion 382 on the base end side relative to the first substrate portion 381. The substrate 38 is formed by folding back an elongated flexible substrate twice. The substrate 38 is folded in the distal end direction Fr at the base end of the first substrate portion 381 and is further folded back in the base end direction Rr, thereby forming a substantially Z-shape. The sensor 37 and the cable 39 are electrically connected to each other by a wiring provided on the substrate 38.

As shown in FIG. 3, the imaging unit 300 comprises a shaft member 33 that is fixed to the movable lens holder 32 and extends toward the base end side relative to the movable lens holder 32, and an actuator 34 that moves the shaft member 33 in the optical axis direction along the optical axis AX.

In the example shown in FIG. 6, the shaft member 33 is disposed on a left side of the fixed lens group and the movable lens group, and the shaft member 33, the fixed lens group, and the movable lens group are provided side by side in the left-right direction. A maximum length 33La of the shaft member 33 in the up-down direction is shorter than a diameter φ of the movable lens 32A (in a case in which the movable lens group includes a plurality of lenses, a maximum diameter of the lenses). In addition, a maximum length 33Lb of the shaft member 33 in the left-right direction is shorter than the diameter φ of the movable lens 32A. With such a configuration, the diameter of the insertion part 2 can be reduced.

In addition, the maximum length 33La of the shaft member 33 is shorter than the maximum length of each of the observation window 30, the first illumination window 24, the second illumination window 22, the forceps port 26, and the air/water supply nozzle 27 in the up-down direction. The maximum length 33Lb of the shaft member 33 is shorter than the maximum length of each of the observation window 30, the first illumination window 24, the second illumination window 22, the forceps port 26, and the air/water supply nozzle 27 in the left-right direction. Since the shaft member 33 is thin as described above, the diameter of the distal end portion 7 can be reduced.

As shown in FIG. 7, the shaft member 33 is inserted into a through-hole 32B provided to penetrate the movable lens holder 32 from the base end side to the distal end side. In the through-hole 32B, the shaft member 33 is fixed to the movable lens holder 32 by press-fitting, welding, adhesion, or the like. A distal end side of the shaft member 33 protrudes toward the distal end side relative to the through-hole 32B and extends toward the distal end side relative to the movable lens holder 32.

The extension portion 711 of the housing member 71 is provided with an insertion hole 711A into which the shaft member 33 is inserted so as to be movable in the optical axis direction. A gap between an inner peripheral surface of the insertion hole 711A and the shaft member 33 has a size such that the shaft member 33 can move in the optical axis direction but cannot move in the up-down direction and the left-right direction. By setting the gap in this way, the position of the shaft member 33 in the directions (up-down direction and left-right direction) perpendicular to the optical axis AX is restricted by the inner peripheral surface of the insertion hole 711A. The inner peripheral surface of the insertion hole 711A forms a first region 331 of a first restriction part that restricts the position of the shaft member 33 in the directions perpendicular to the optical axis AX. The maximum lengths 33La and 33Lb shown in FIG. 6 are lengths (maximum values of widths in the left-right direction and the up-down direction) of portions of the shaft member 33 inserted into the insertion hole 711A.

By restricting the position of the shaft member 33 in the left-right direction, the position of the movable lens holder 32 fixed to the shaft member 33 in the left-right direction can be held in a desired state, and the movable lens group can be prevented from tilting in the optical axis direction. The first region 331 can be said to be a region that, in a side view in the up-down direction, restricts a position of the movable lens holder 32 in the left-right direction and a relative angle between the optical axis AX and the movable lens holder 32 (angle formed between the optical axis of the movable lens 32A and the optical axis AX, and a tilt angle of the movable lens group) and comes into contact with the shaft member 33.

By increasing the length LA (same meaning as the length between the distal end and the base end of the first restriction part) of the first region 331 in the optical axis direction, the effect of preventing the movable lens group from tilting can be enhanced. For example, the effect of preventing the tilting can be enhanced by making the length LA longer than the length LB (in a case in which the movable lens group includes a plurality of lenses, a minimum value thereof) in the optical axis direction of the movable lens 32A. In addition, by making the length LA longer than the diameter φ of the movable lens 32A (in a case in which the movable lens group includes a plurality of lenses, a minimum value thereof), the effect of preventing the tilting can be enhanced.

By increasing the length of the portion of the shaft member 33 inserted into the insertion hole 711A, the effect of preventing the tilting can be further enhanced. For example, by providing the base end of the shaft member 33 on the base end side relative to a light incident surface 36A of the prism 36, the effect of preventing the tilting can be enhanced. In addition, by providing the base end of the shaft member 33 on the base end side relative to the light-receiving surface of the sensor 37, the effect of preventing the tilting can be further enhanced.

A separate member that fills the gap between the insertion hole 711A and the shaft member 33 may be provided in the insertion hole 711A to restrict the position of the shaft member 33 in the left-right direction.

In the endoscope 1, the fixed lens holder 31, the left light guide 220, the right light guide 240, the treatment tool pipe line 260, and the fluid pipe line 270 are fixed to the housing member 71. In this way, the first region 331 is formed by using a part of the housing member 71 having a function of housing the internal member assembly OB, so that the diameter of the distal end portion 7 can be reduced.

The actuator 34 is provided on the base end side of the shaft member 33, and an end surface 340Fr (see FIG. 7) of the actuator 34 on the distal end side is in contact with an end surface 33Rr (see FIG. 7) of the shaft member 33 on the base end side. The actuator 34 includes a motor and a movable part that is movable in the optical axis direction by the rotation of the motor, and moves the shaft member 33 by the movement of the movable part. As the shaft member 33 is moved, the movable lens 32A held by the movable lens holder 32 moves in a movement region R1 shown in FIG. 7. The first region 331 of the shaft member 33 is located on the base end side relative to a base end of the movement region R1.

FIG. 7 shows the actuator 34 including a sliding screw 340 as a movable part and a flexible shaft 341 connected to the sliding screw 340. The sliding screw 340 is inserted into the insertion hole provided in the extension portion 711. The flexible shaft 341 is connected to the motor provided in the operating part 3. In a case in which the motor rotates, the flexible shaft 341 and the sliding screw 340 connected to the flexible shaft 341 rotate, and the sliding screw 340 moves in the optical axis direction.

As shown in FIG. 7, it is preferable that a length LAa of the portion of the shaft member 33 on the base end side relative to the movable lens holder 32 is longer than the length LA of the first region 331. In this manner, the shaft member 33 can be pressed by the actuator 34 outside the insertion hole 711A. As a result, the restriction on the position of the actuator 34 in the left-right direction can be relaxed. For example, as shown in FIG. 7, a distance L6 between a centroid 34C of the actuator 34 and the optical axis AX can be made shorter than a distance L7 between a centroid 33C of the shaft member 33 and the optical axis AX. That is, the actuator 34 can be disposed closer to the optical axis AX than the shaft member 33, and thus the diameter of the distal end portion 7 can be reduced.

In addition, as shown in FIG. 7, a configuration can also be adopted in which one end E1, on the optical system OP side, of the end surface 33Rr of the shaft member 33 is located on a side opposite to the optical system OP side relative to one end E2, on the optical system OP side, of the end surface 340Fr of the actuator 34, and the other end E3, on a side opposite to the optical system OP side, of the end surface 33Rr of the shaft member 33 is located on a side opposite to the optical system OP side relative to the other end E4, on a side opposite to the optical system OP side, of the end surface 340Fr of the actuator 34. According to this configuration, the diameter of the distal end portion 7 can be reduced.

As shown in FIGS. 6 and 7, in the fixed lens holder 31, a protrusion 310 that protrudes in the left direction L from a region in which the fixed lens group is housed is provided. The protrusion 310 overlaps the distal end of the shaft member 33 in a view in the optical axis direction. A compression spring 35 as a biasing member is provided around the distal end side of the shaft member 33 in a state in which an initial tension is applied. The compression spring 35 biases the movable lens holder 32 toward the base end side. In this way, by biasing the movable lens holder 32 toward the base end side, the contact property between the shaft member 33 and the actuator 34 is improved, and the movement accuracy of the shaft member 33 by the actuator 34 can be improved.

In the example shown in FIG. 7, the end surface 33Rr of the shaft member 33 and the end surface 340Fr of the actuator 34 are in contact with each other in a state in which the movable lens 32A is located at the base end of the movement region R1, but, in this state, the end surface 33Rr and the end surface 340Fr need not be in contact with each other.

For example, in FIG. 7, the sliding screw 340 may be in a state of being slightly moved rearward. As described above, even in a case in which the end surface 33Rr and the end surface 340Fr are not in contact with each other in the state shown in FIG. 7, the movable lens holder 32 is pressed against the extension portion 711 by the biasing force of the compression spring 35, and thus the position of the movable lens holder 32 can be held with high accuracy.

According to this configuration, in the state shown in FIG. 7, since it is not necessary to move the sliding screw 340 to the position at which the sliding screw 340 abuts on the shaft member 33, it is easy to control the actuator 34 and the like. In addition, the movable lens 32A can be positioned at the base end of the movement region R1 with high accuracy by the contact between the movable lens holder 32 and the extension portion 711. Meanwhile, in a case in which the movable lens 32A is moved to a position other than the base end of the movement region R1, the position of the movable lens 32A can be determined with high accuracy by bringing the shaft member 33 and the sliding screw 340 into contact with each other.

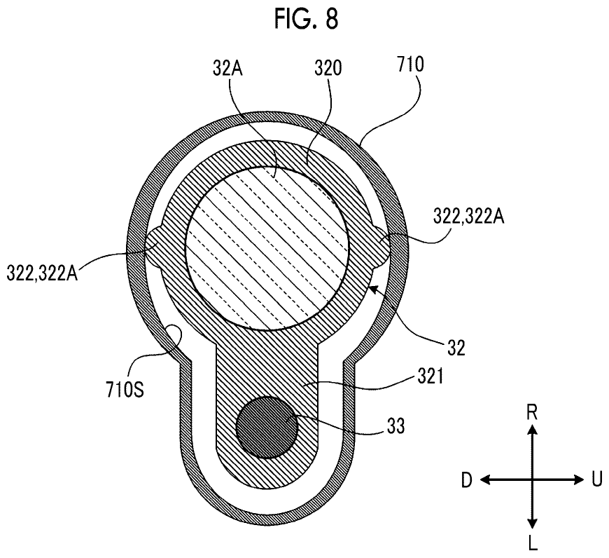

FIG. 8 is a schematic cross-sectional view taken along line B-B shown in FIG. 7. As shown in FIG. 8, the shaft member 33 has a circular cross section. The movable lens holder 32 comprises an annular region 320 that houses the movable lens 32A and a linear region 321 that extends from the region 320 in the left direction L, and the shaft member 33 is inserted into and fixed in the region 321.

The movable lens holder 32 further comprises a second restriction part 322 that restricts rotation of the movable lens holder 32 about the axis of the shaft member 33, in other words, rotation of the movable lens holder 32 in the up-down direction.

The second restriction part 322 is provided around the region 320, and is configured with a protrusion 322A that protrudes in the up direction U from an upper end portion of the region 320 and a protrusion 322A that protrudes in the down direction D from a lower end portion of the region 320. A gap between the second restriction part 322 and an inner peripheral surface 710S of the body portion 710 is set to a size such that the movable lens holder 32 can move in the optical axis direction but the movable lens holder 32 cannot rotate. By providing the second restriction part 322, the movable lens group can be prevented from moving in the up-down direction.

A length of the second restriction part 322 in the optical axis direction is shorter than a length of the first region 331 in the optical axis direction. In other words, the length of the first region 331 in the optical axis direction is longer than the length of the second restriction part 322 in the optical axis direction.

In the endoscope 1, since the first region 331 of the first restriction part prevents the movable lens group from tilting in the optical axis direction and the second restriction part 322 prevents the movable lens group from rotating in the up-down direction, the structure of each restriction part can be simplified. In addition, by providing the restriction part in two parts, the length of the second restriction part 322 in the optical axis direction can be made shorter than the length of the first region 331. As a result, a space in the distal end portion 7 can be efficiently used, and the diameter can be reduced.

In addition, the first region 331 and the second restriction part 322 are provided at different positions in the left-right direction. Therefore, a space in the distal end portion 7 can be efficiently used, and the diameter can be reduced. In addition, in the left-right direction, the first region 331 is located in a different region from the movable lens group, and the second restriction part 322 is located closer to the movable lens group than the first region 331 (preferably, in a region overlapping the movable lens group). As described above, by preventing the movable lens group from rotating in the up-down direction at a position close to the lens, it is possible to simplify the structure of the second restriction part 322.

As shown in FIG. 6, it is preferable that the shaft member 33 is located on the observation window 30 side relative to the first illumination window 24. That is, it is preferable that a distance between the centers of the shaft member 33 and the observation window 30 is shorter than a distance between the centers of the first illumination window 24 and the observation window 30.

In addition, it is preferable that the shaft member 33 is located on an outer edge side of the distal end surface 14 relative to the first illumination window 24 and the observation window 30. That is, it is preferable that a distance L5 between a center CP of the distal end surface 14 and the center of the shaft member 33 is longer than a distance L1 between the center CP of the distal end surface 14 and the center of the first illumination window 24, and is longer than a distance L4 between the center CP of the distal end surface 14 and the center of the observation window 30.

In addition, it is preferable that the shaft member 33 is located on a side opposite to the air/water supply nozzle 27 with respect to a line DL connecting the centers of the observation window 30 and the forceps port 26.

In addition, it is preferable that the distance L5 from the center CP to the center of the shaft member 33 is longer than a distance (distance L1, distance L2, distance L3, distance L4) from the center CP to each functional element. That is, the center of the shaft member 33 may be located on the outer edge side of the distal end surface 14 relative to the center of the observation window 30 and the centers of the other functional elements.

According to these configurations, the diameter of the distal end portion 7 can be reduced.

In addition, as shown in FIG. 6, the center of the shaft member 33 may be located in a region interposed between a line extending from the center CP of the distal end surface 14 and passing through the center of the observation window 30 and a line extending from the center CP of the distal end surface 14 and passing through the center of each functional element other than the observation window 30. In the example of FIG. 6, the center of the shaft member 33 is located in a region interposed between a line extending from the center CP of the distal end surface 14 and passing through the center of the observation window 30 and a line extending from the center CP of the distal end surface 14 and passing through the center of the second illumination window 22. The technology of the present disclosure is not limited to this example, and, for example, the center of the shaft member 33 may be located in a region interposed between a line extending from the center CP of the distal end surface 14 and passing through the center of the observation window 30 and a line extending from the center CP of the distal end surface 14 and passing through the center of the first illumination window 24.

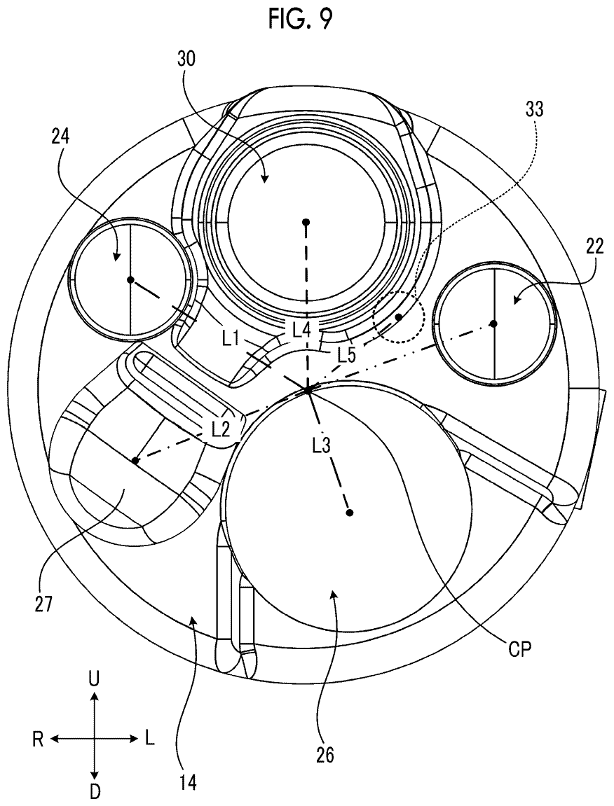

FIG. 9 is a schematic view showing a modification example of the disposition of the shaft member 33. As shown in FIG. 9, the center of the shaft member 33 may be located on the center CP side of the distal end surface 14 relative to the center of the observation window 30 and the centers of the other functional elements. That is, the distance L5 may be shorter than the distances L1 to L4.

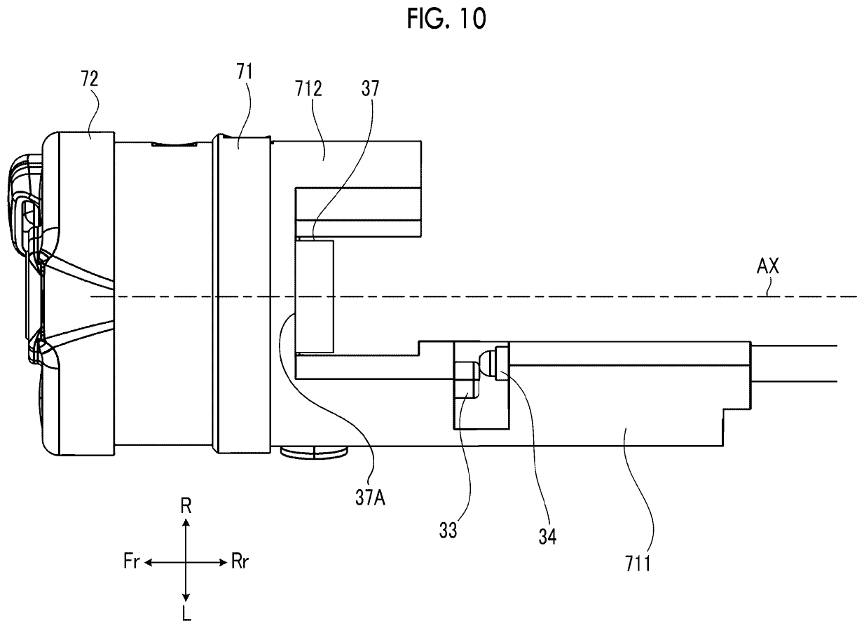

The sensor 37 is configured such that the light-receiving surface is disposed along the optical axis AX, but the technology of the present disclosure is not limited to this. For example, as shown in FIG. 10, a configuration can also be adopted in which the prism 36 is removed and the sensor 37 provided with the light-receiving surface 37A disposed perpendicular to the optical axis AX at a position of the prism 36. With this configuration, it is preferable that the sensor 37 and the actuator 34 overlap each other in a state of being viewed from the distal end side. As a result, the diameter of the distal end portion 7 can be reduced.

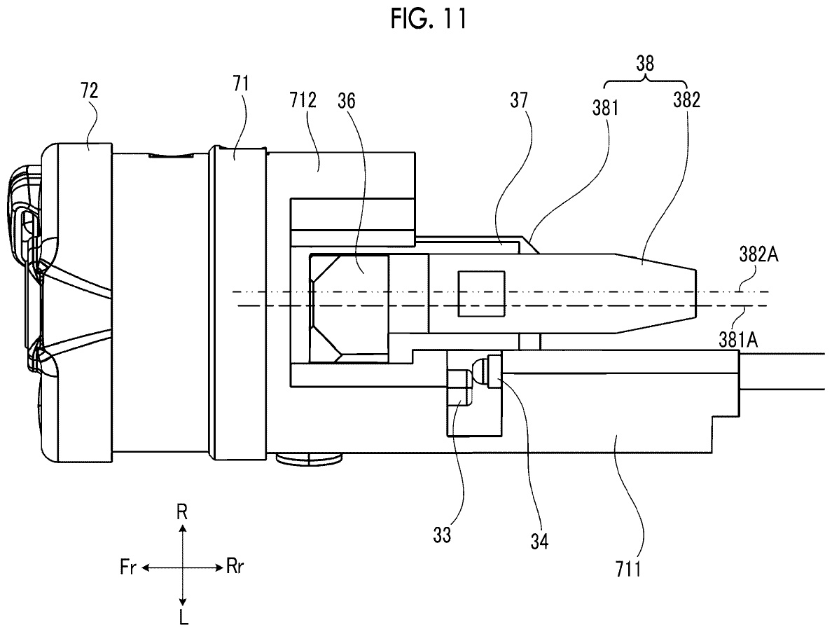

FIG. 11 is a schematic view showing a modification example of the substrate 38. In the configuration shown in FIG. 11, a center 382A of the second substrate portion 382 in the direction (left-right direction) along a short side of the sensor 37 is shifted, relative to a center 381A of the first substrate portion 381 in the left-right direction, in a direction away from the shaft member 33 and the actuator 34. According to this configuration, a space between the second substrate portion 382 and the extension portion 711 can be effectively utilized, and the diameter can be reduced. In addition, the prism 36 and the actuator 34 can be disposed so as to overlap each other in the axial direction, and the diameter of the distal end portion 7 can be reduced.

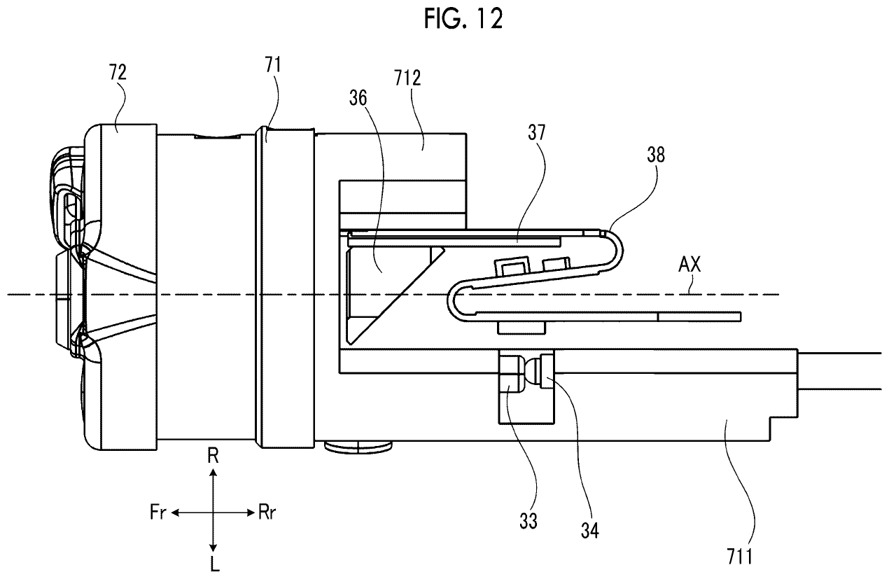

FIG. 12 is a schematic diagram showing a modification example of the disposition of the prism 36, the sensor 37, and the substrate 38. In the configuration shown in FIG. 12, the prism 36, the sensor 37, and the substrate 38 are rotated about the optical axis AX by 90 degrees toward the front of the paper surface with respect to the configuration shown in FIG. 5. In the configuration shown in FIG. 12, the shaft member 33 is provided on a side opposite to the sensor 37 with a plane that passes through the optical axis AX and that is parallel to the light-receiving surface 37A of the sensor 37 interposed therebetween. In this configuration as well, the diameter can be reduced by the technology of the present disclosure.

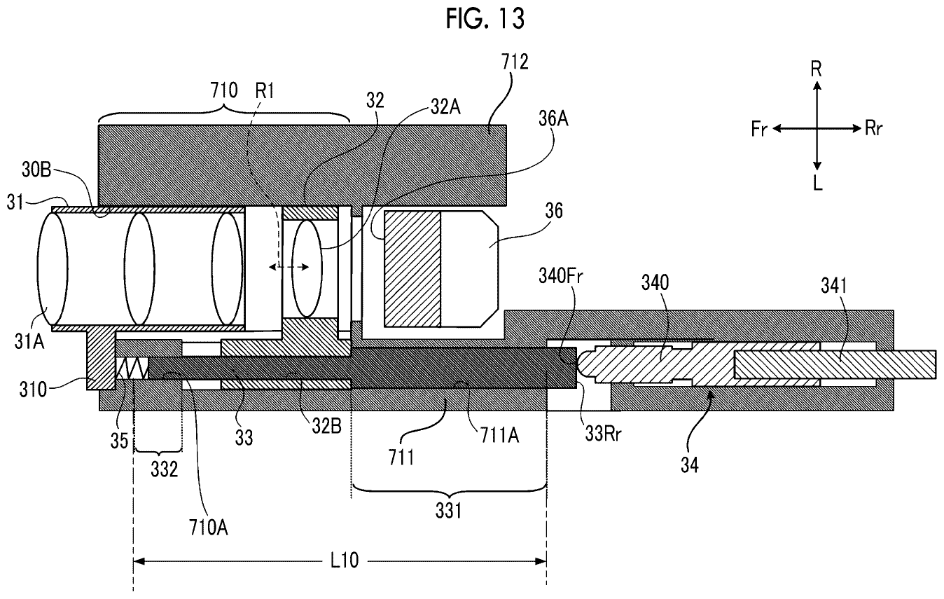

FIG. 13 is a schematic view showing a first modification example of the imaging unit 300 and the housing member 71, and is a view taken along line A-A shown in FIG. 6. The configuration shown in FIG. 13 is different from the configuration shown in FIG. 7 in that a distal end region of the shaft member 33 extending toward the distal end side relative to the movable lens holder 32 is inserted into an insertion hole 710A formed in the body portion 710.

A gap between an inner peripheral surface of the insertion hole 710A and the distal end region of the shaft member 33 has a size such that the shaft member 33 can move in the optical axis direction but cannot in the up-down direction and the left-right direction. Since the gap is set as described above, the position of the shaft member 33 in the up-down direction and the left-right direction is restricted by the inner peripheral surface of the insertion hole 710A. The second region 332 of the first restriction part that restricts the position of the shaft member 33 in the directions perpendicular to the optical axis AX is formed by the inner peripheral surface of the insertion hole 710A. The second region 332 of the shaft member 33 is located on the distal end side relative to the distal end of the movement region R1.

As described above, by providing the second region 332 in addition to the first region 331, it is possible to enhance the effect of preventing the movable lens group from tilting. In a case in which a length L10 between the base end of the first restriction part (base end of the first region 331) and the distal end of the first restriction part (distal end of the second region 332) in the optical axis direction is made longer than the length LB of the movable lens 32A in the optical axis direction, the effect of preventing the tilting can be enhanced. In addition, by making the length L10 longer than the diameter φ of the movable lens 32A, the effect of preventing the tilting can be enhanced. By providing the second region 332 on the distal end side of the movable lens holder 32, the length of the first region 331 can be shortened, and thus a space in the optical axis direction can be reduced.

A separate member that fills the gap between the insertion hole 710A and the shaft member 33 may be provided in the insertion hole 710A to restrict the position of the shaft member 33.

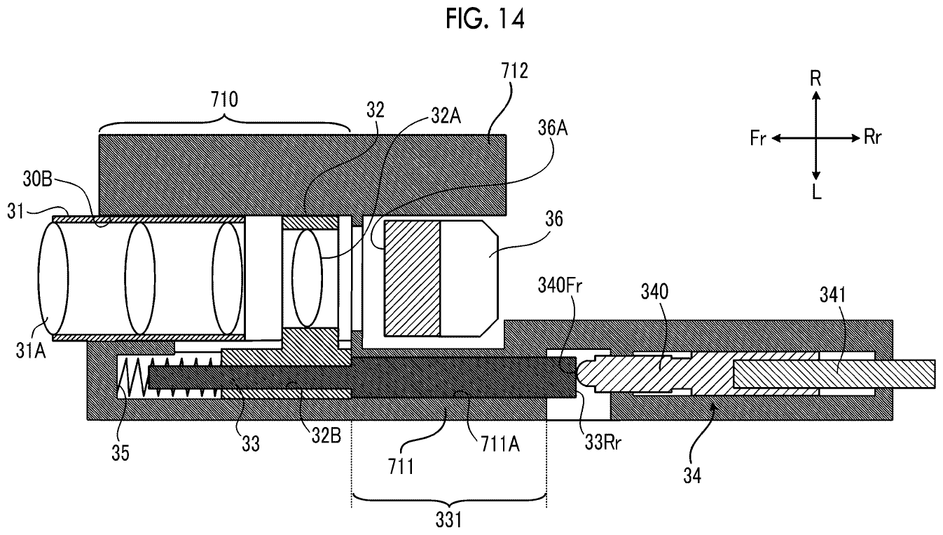

FIG. 14 is a schematic view showing a second modification example of the imaging unit 300 and the housing member 71, and is a view taken along line A-A shown in FIG. 6. In the example shown in FIG. 14, the configuration is different from the configuration shown in FIG. 7 in that the protrusion 310 in the fixed lens holder 31 is removed, a wall is formed by a part of the body portion 710 at a position facing the distal end of the shaft member 33, and the compression spring 35 is provided between the wall and the shaft member 33. According to the configuration shown in FIG. 14, the assembly precision of the distal end portion 7 can be improved.

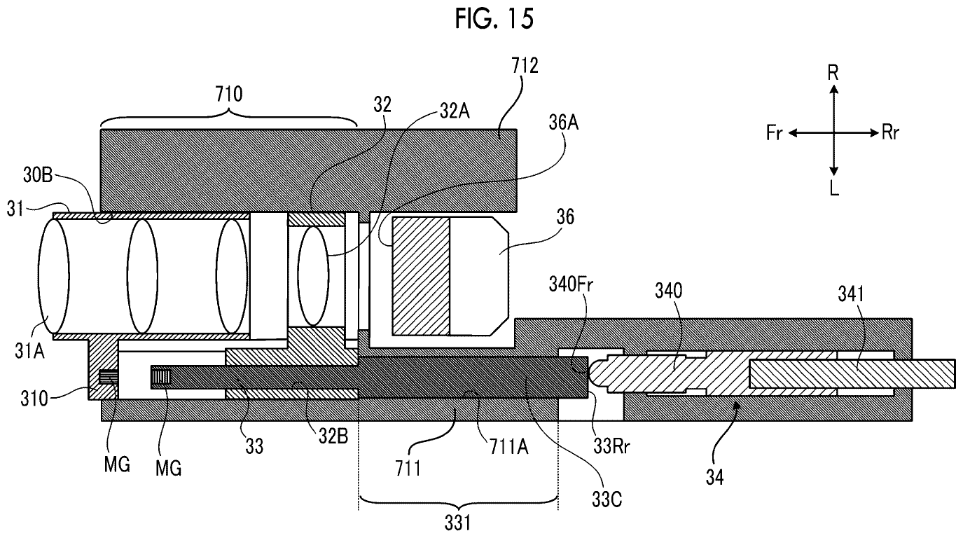

FIG. 15 is a schematic view showing a third modification example of the imaging unit 300 and the housing member 71, and is a view taken along line A-A shown in FIG. 6. In the example shown in FIG. 15, the configuration is different from the configuration shown in FIG. in that two magnets MG are provided instead of the compression spring 35.

One of the two magnets MG is provided at the protrusion 310, and the other of the two magnets MG is provided at the distal end of the shaft member 33. The polarities of the two magnets MG are set to repel each other. As a result, the shaft member 33 is biased toward the actuator 34. According to the configuration shown in FIG. 15, the durability can be improved as compared with a case in which a spring is used as a biasing unit.

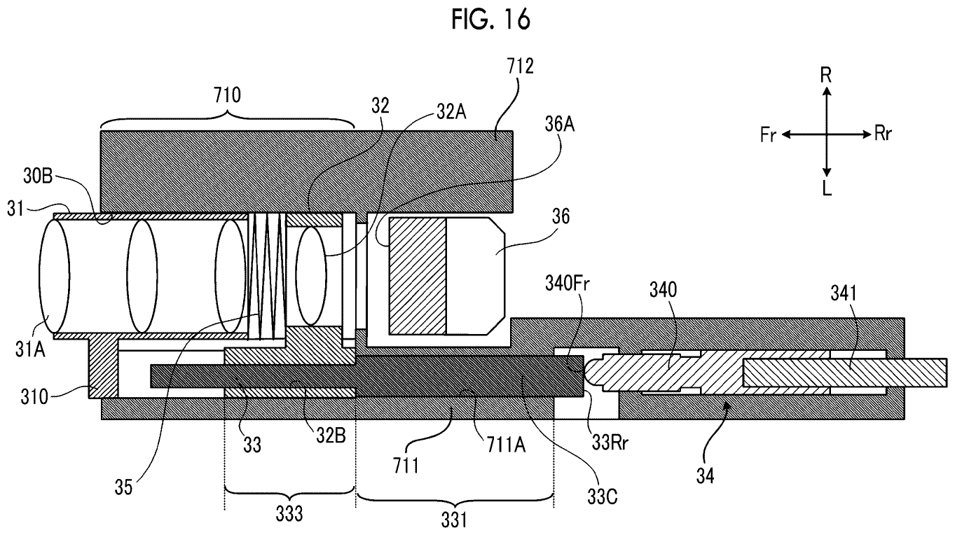

FIG. 16 is a schematic view showing a fourth modification example of the imaging unit 300 and the housing member 71, and is a view taken along line A-A shown in FIG. 6. In the example shown in FIG. 16, the configuration is different from the configuration shown in FIG. 7 in that the position of the compression spring 35 is changed to a position between the fixed lens holder 31 and the movable lens holder 32.

The compression spring 35 biases the movable lens holder 32 toward the base end side. According to the configuration shown in FIG. 16, since the compression spring 35 is disposed in a space to be provided between the fixed lens holder 31 and the movable lens holder 32, it is possible to reduce the size of the distal end portion 7.

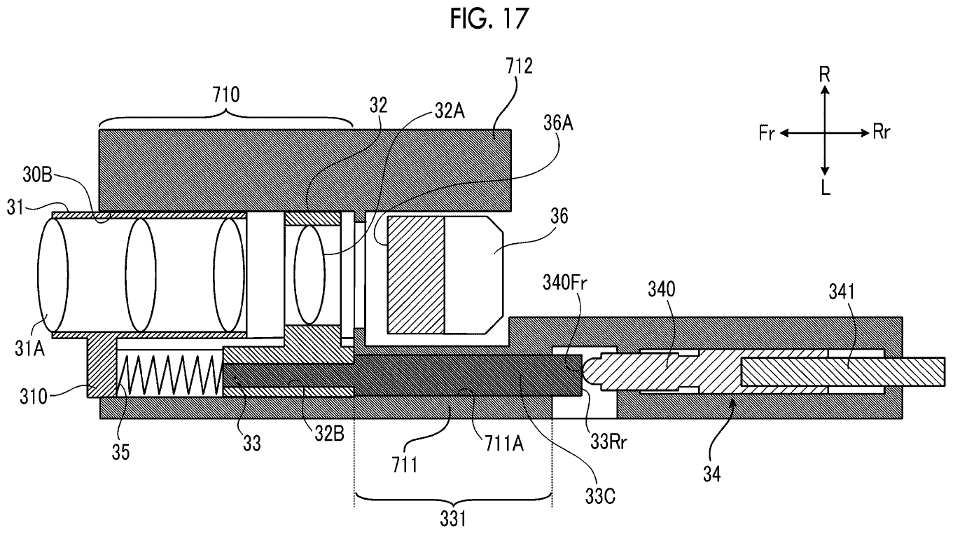

FIG. 17 is a schematic view showing a fifth modification example of the imaging unit 300 and the housing member 71, and is a view taken along line A-A shown in FIG. 6. In the example shown in FIG. 17, the configuration is different from the configuration shown in FIG. 7 in that the shaft member 33 does not have a region that extends toward the distal end side relative to the movable lens holder 32. In the configuration shown in FIG. 17 as well, the first region 331 is provided, and thus the effect of preventing the movable lens group from tilting can be obtained. In addition, according to the configuration shown in FIG. 17, the structure can be simplified as compared with the configuration shown in FIG. 7, and it is possible to reduce the manufacturing cost or to achieve size reduction.

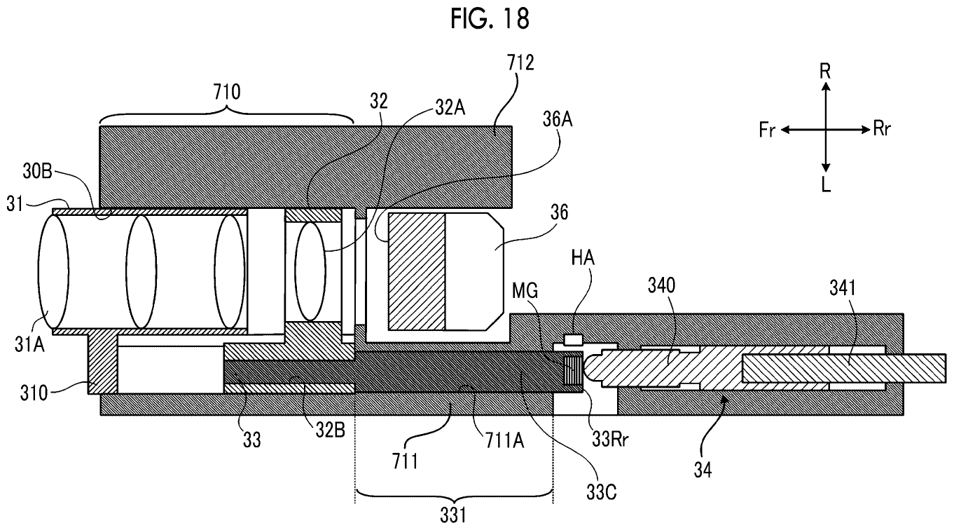

FIG. 18 is a schematic view showing a sixth modification example of the imaging unit 300 and the housing member 71, and is a view taken along line A-A shown in FIG. 6. In the example shown in FIG. 18, the configuration is different from the configuration shown in FIG. 7 in that the shaft member 33 does not have the region extending toward the distal end side relative to the movable lens holder 32, the compression spring 35 is removed and the magnet MG is provided at the base end of the shaft member 33 instead, and the Hall element HA as a magnetic detection element is added.

The Hall element HA is provided on the extension portion 711 in the vicinity of the magnet MG. The sliding screw 340 is made of a magnetic material. Therefore, the magnet MG is attracted to the sliding screw 340. As a result, the movable lens holder 32 and the shaft member 33 are biased toward the actuator 34. The magnet MG and the Hall element HA form a position detection unit that detects the position of the shaft member 33 in the optical axis direction. As described above, by using the magnet MG as a biasing unit as the position detection unit, it is possible to reduce the number of components. In addition, since the compression spring 35 on the distal end side is removed, it is possible to achieve size reduction.

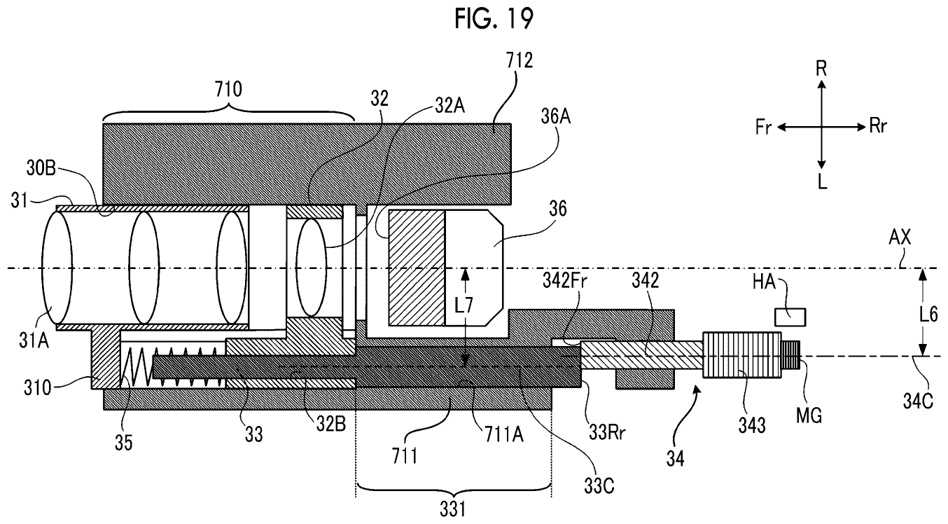

FIG. 19 is a schematic view showing a seventh modification example of the imaging unit 300 and the housing member 71, and is a view taken along line A-A shown in FIG. 6. In the example shown in FIG. 19, the configuration of the actuator 34 is different from the configuration shown in FIG. 7.

In the example shown in FIG. 19, the actuator 34 includes a piezoelectric element 343 and a rod 342 that is operable in the optical axis direction by the operation of the piezoelectric element 343, and the shaft member 33 moves by the movement of the rod 342. The piezoelectric element 343 is configured such that, for example, a plurality of piezoelectric elements 343 are stacked, and is expandable and contractible in the optical axis direction. The rod 342 is fixed to the piezoelectric element 343 and extends toward the distal end side relative to the piezoelectric element 343. An end surface 342Fr of the rod 342 on the distal end side is in contact with the end surface 33Rr of the shaft member 33. The rod 342 is held by the extension portion 711 with moderate friction. For example, the rod 342 is held by the extension portion 711 by a leaf spring and the like. The magnet MG is provided on a base end side of the piezoelectric element 343, and the hall element HA is provided in the vicinity of the magnet MG.

A magnitude relationship between the distance L6 between the centroid 34C of the actuator 34 and the optical axis AX and the distance L7 between the centroid 33C of the shaft member 33 and the optical axis AX is the same as the magnitude relationship shown in FIG. 7. In addition, the configuration in which the right end of the end surface 33Rr of the shaft member 33 is located on the left side relative to the right end of the end surface 342Fr of the actuator 34 and the left end of the end surface 33Rr of the shaft member 33 is located on the left side relative to the left end of the end surface 342Fr of the actuator 34 is also the same as the configuration shown in FIG. 7.

By repeatedly executing an operation in which the piezoelectric element 343 extends slowly and contracts rapidly, or vice versa, the rod 342 and the extension portion 711 alternately slip and stop, causing the piezoelectric element 343 and the rod 342 to move in the optical axis direction and thereby moving the shaft member 33 and the movable lens holder 32 along the optical axis. The magnet MG and the Hall element HA form a position detection unit that detects the position of the shaft member 33 in the optical axis direction. According to the configuration shown in FIG. 19, it is not necessary to extend the flexible shaft to the entire insertion part 2 as compared with the configuration shown in FIG. 7, and thus the structure of the insertion part 2 can be simplified.

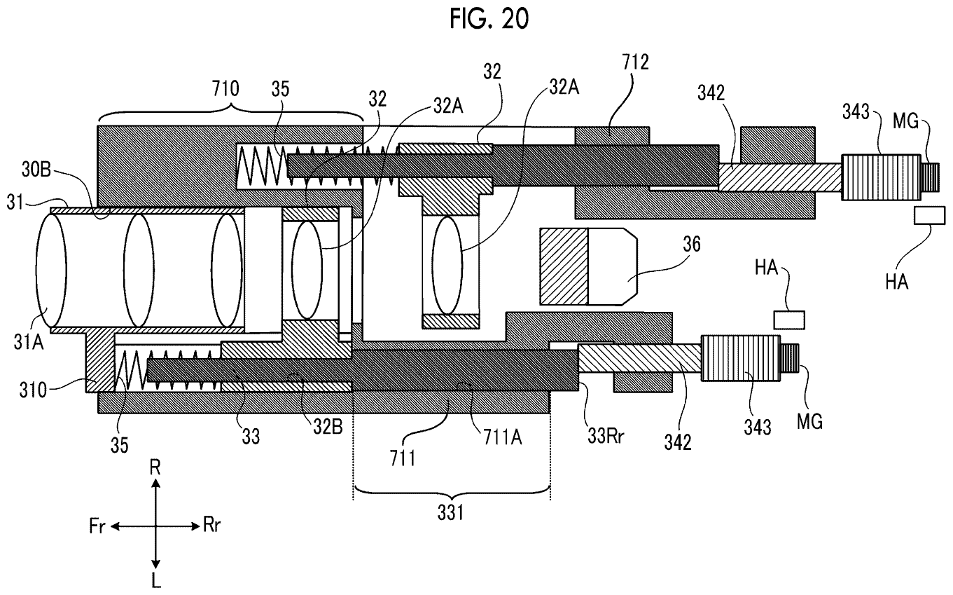

FIG. 20 is a schematic view showing an eighth modification example of the imaging unit 300 and the housing member 71, and is a view taken along line A-A shown in FIG. 6. FIG. 20 shows a configuration in which one set of the movable lens 32A, the movable lens holder 32, the shaft member 33, the compression spring 35, and the actuator 34 is added to the configuration shown in FIG. 19. As shown in FIG. 20, the technology of the present disclosure can also be applied to a case in which a plurality of movable lenses 32A are individually moved.

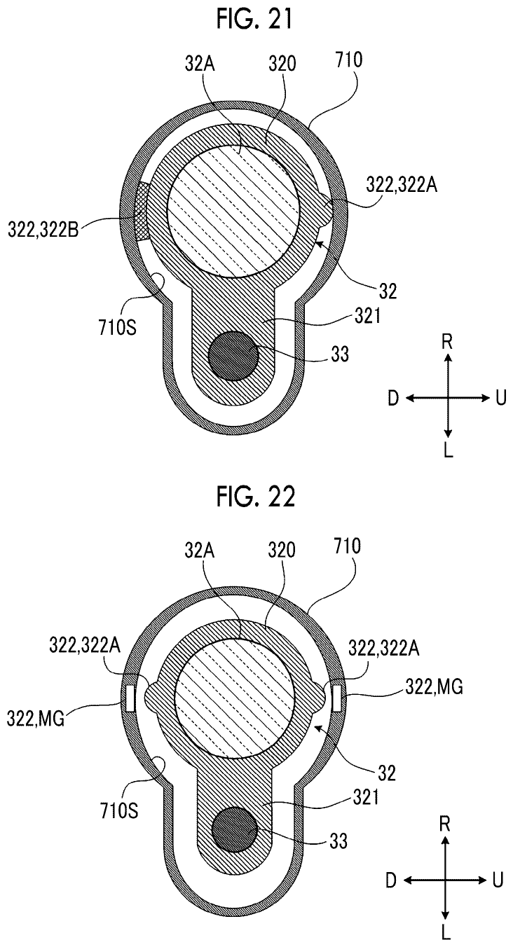

FIG. 21 is a schematic view showing a first modification example of a second restriction part 322, and is a view taken along line B-B shown in FIG. 7. The example shown in FIG. 21 shows a configuration in which one of the two protrusions 322A shown in FIG. 8 is removed, and instead, a member 322B that fills a gap between an outer peripheral surface of the movable lens holder 32 and the body portion 710 is added to the outer peripheral surface. A gap between the member 322B and the protrusion 322A and the body portion 710 is set to such an extent that the movable lens holder 32 can move in the optical axis direction and the movable lens holder 32 cannot rotate about the axis of the shaft member 33. In FIG. 21, the protrusion 322A may be replaced with the member 322B.

FIG. 22 is a schematic view showing a second modification example of the second restriction part 322, and is a view taken along line B-B shown in FIG. 7. In the example shown in FIG. 22, the configuration is different from the configuration shown in FIG. 8 in that the magnet MG is provided at a position facing the protrusion 322A on the inner periphery of the body portion 710, and the protrusion 322A is made of at least a magnetic material.

In the example shown in FIG. 22, a gap between the protrusion 322A and the body portion 710 may be wide to the extent that the movable lens holder 32 can rotate in the up-down direction. In the example of FIG. 22, the rotation of the movable lens holder 32 in the up-down direction is restricted by a repulsive force between the magnet MG and the protrusion 322A. In this way, the protrusion 322A and the magnet MG can also form the second restriction part 322.

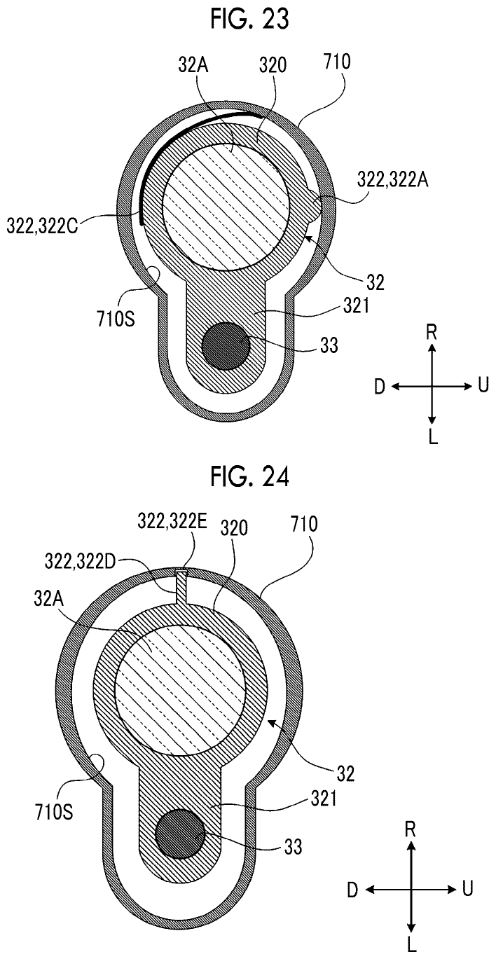

FIG. 23 is a schematic view showing a third modification example of the second restriction part 322, and is a view taken along line B-B shown in FIG. 7. The example shown in FIG. 23 shows a configuration in which one of the two protrusions 322A shown in FIG. 8 is removed, and instead, a leaf spring 322C that fills a gap between an outer peripheral surface of the movable lens holder 32 and the body portion 710 is added to the outer peripheral surface. The leaf spring 322C extends to the right side from the same position as the protrusion 322A in the left-right direction along the outer periphery of the region 320, the right end of the leaf spring 322C is fixed to the inner peripheral surface 710S, the left end of the leaf spring 322C comes into contact with the region 320 in the vicinity of the position facing the protrusion 322A, and the leaf spring 322C biases the region 320 toward the protrusion 322A.

According to the configuration shown in FIG. 23, the effect of preventing the movable lens group from rotating in the up-down direction can be enhanced.

FIG. 24 is a schematic view showing a fourth modification example of the second restriction part 322, and is a view taken along line B-B shown in FIG. 7. In the example shown in FIG. 24, in the configuration shown in FIG. 8, two protrusions 322A are removed, a protrusion 322D that protrudes in the right direction R is provided at the right end of the region 320, and a recessed portion 322E that receives the protrusion 322D movably in the optical axis direction is provided in the body portion 710. In a case in which the protrusion 322D is inserted into the recessed portion 322E, the rotation of the movable lens holder 32 in the up-down direction is restricted. The protrusion 322D and the recessed portion 322E form the second restriction part 322.

In the example shown in FIG. 24, the second restriction part 322 is located on a side opposite to the first restriction part (first region 331) side relative to the optical axis AX in the left-right direction. According to such a configuration, it is possible to enhance the effect of preventing the movable lens group from rotating in the up-down direction.

In the above description, the housing member 71 houses the imaging unit 300 and houses the left light guide 220, the right light guide 240, the treatment tool pipe line 260, and the fluid pipe line 270, but the technology of the present disclosure is not limited to this. A housing member that houses the imaging unit 300 and a housing member that houses the left light guide 220, the right light guide 240, the treatment tool pipe line 260, and the fluid pipe line 270 may be provided independently of each other.

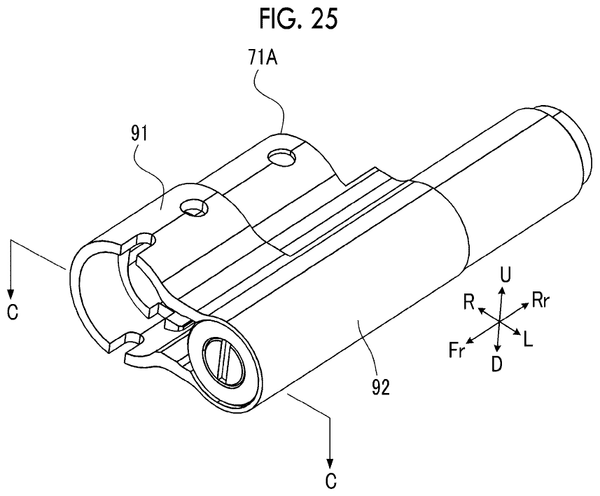

FIG. 25 is a perspective view showing an external configuration of a housing member 71A that houses the imaging unit 300 of the modification example. The housing member 71A comprises a first housing region 91 that houses the lens of the optical system OP, and a second housing region 92 that houses a mechanism for moving the movable lens included in the optical system OP.

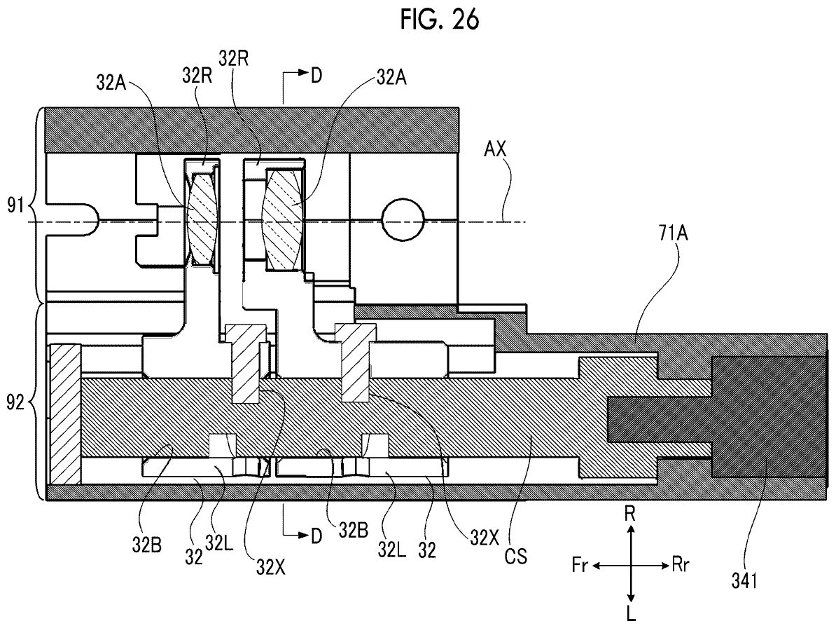

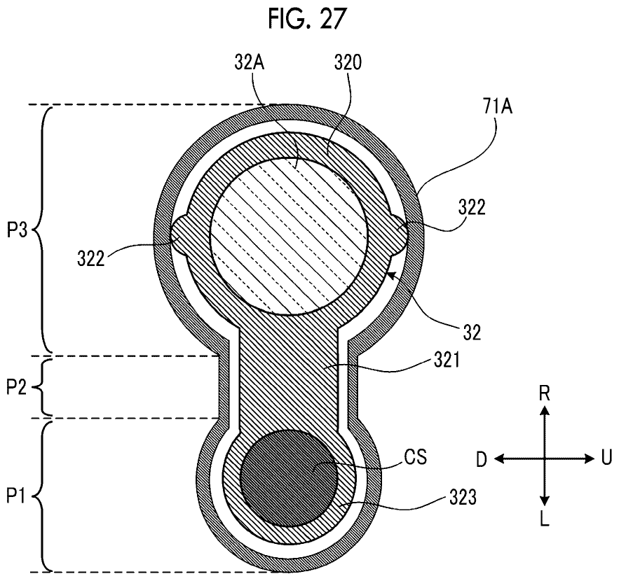

FIG. 26 is a schematic cross-sectional view taken along line C-C shown in FIG. 25. The optical system OP is provided with two movable lenses 32A. The fixed lens group housed in the first housing region 91 is not shown.

Two movable lens holders 32 that hold the movable lens 32A are provided inside the housing member 71A so as to be arranged in the optical axis direction. The movable lens holder 32 comprises a region 32R that houses the movable lens 32A and a region 32L that is located on the left side of the region 32R. A length of the region 32L in the optical axis direction is longer than a length of the region 32R in the optical axis direction.

A camshaft CS that rotates by the rotation of the flexible shaft 341 connected to the motor is provided inside the second housing region 92 so as to extend in the optical axis direction. The region 32L of the movable lens holder 32 is provided with a through-hole 32B through which the camshaft CS passes. An engaging protrusion 32X that engages with a cam groove provided in the camshaft CS is provided in the region 32L of the movable lens holder 32. In a case in which the flexible shaft 341 rotates by the operation of the motor, the camshaft CS rotates about the rotation axis extending in the optical axis direction. As a result, the engaging protrusion 32X moves along the cam groove, and the movable lens holder 32 moves in the optical axis direction. A gap between an inner peripheral surface of the through-hole 32B and the camshaft CS is set to be small to the extent that the movable lens holder 32 can move in the optical axis direction and cannot move in the up-down direction and the left-right direction.

In the configuration shown in FIG. 26, the camshaft CS forms a shaft member that extends toward the base end side relative to the movable lens holder 32 and is connected to the movable lens holder 32 and the actuator (flexible shaft 341) to move the movable lens holder 32 in the optical axis direction. In addition, the inner peripheral surface of the through-hole 32B forms a first restriction part that restricts the position of the movable lens holder 32 in the left-right direction and the relative angle between the optical axis AX and the movable lens holder 32, and comes into contact with the shaft member. A member that fills the gap between the through-hole 32B and the camshaft CS such that the camshaft CS can rotate but cannot move in the up-down direction and the left-right direction can also be provided, and this member can be used as the first restriction part.

FIG. 27 is a schematic cross-sectional view taken along line D-D shown in FIG. 26. As shown in FIG. 27, the housing member 71A comprises a first portion P1 that houses the through-hole 32B that forms the first restriction part, a second portion P2 that is narrower in the up-down direction than the first portion P1, and a third portion P3 that is wider in the up-down direction than the second portion P2. In the configuration shown in FIG. 8, the configuration of the housing member 71A shown in FIG. 27 may be adopted.

As shown in FIG. 27, the camshaft CS has a circular cross section. The movable lens holder 32 comprises the annular region 320 that houses the movable lens 32A, an annular region 323 into which the camshaft CS is inserted, and the linear region 321 that connects the region 320 and the region 323. The movable lens holder 32 further comprises the second restriction part 322 that restricts the rotation of the movable lens holder 32 about the axis of the camshaft CS, in other words, the rotation of the movable lens holder 32 in the up-down direction.

The second restriction part 322 is provided around the region 320, and is configured with a protrusion 322A that protrudes in the up direction U from an upper end portion of the region 320 and a protrusion 322A that protrudes in the down direction D from a lower end portion of the region 320. The gap between the second restriction part 322 and the inner peripheral surface 710S of the body portion 710 is set to a size such that the movable lens holder 32 can move in the optical axis direction but the movable lens holder 32 cannot rotate. By providing the second restriction part 322, the movable lens group can be prevented from moving in the up-down direction.

The length of the second restriction part 322 in the optical axis direction is shorter than the length of the inner peripheral surface of the through-hole 32B in the optical axis direction.

In the configurations shown in FIGS. 25 to 27, since the first restriction part (inner peripheral surface of the through-hole 32B) prevents the movable lens group from tilting in the optical axis direction and the second restriction part 322 prevents the movable lens group from rotating in the up-down direction, the structures of the restriction parts can be simplified. In addition, by providing the restriction part in two parts, the length of the second restriction part 322 in the optical axis direction can be made shorter than the length of the first restriction part. As a result, a space in the distal end portion 7 can be efficiently used, and thus the size can be reduced.

In addition, the first restriction part and the second restriction part 322 are provided at different positions in the left-right direction. Therefore, a space in the distal end portion 7 can be efficiently used, and the diameter can be reduced. In addition, in the left-right direction, the first restriction part is located in a different region from the movable lens group, and the second restriction part 322 is located closer to the movable lens group than the first region 331 (preferably, in a region overlapping the movable lens group). As described above, by preventing the movable lens group from rotating in the up-down direction at a position close to the lens, it is possible to simplify the structure of the second restriction part 322.

The second restriction part 322 shown in FIG. 27 can be replaced with the configuration shown in FIGS. 21 to 24.

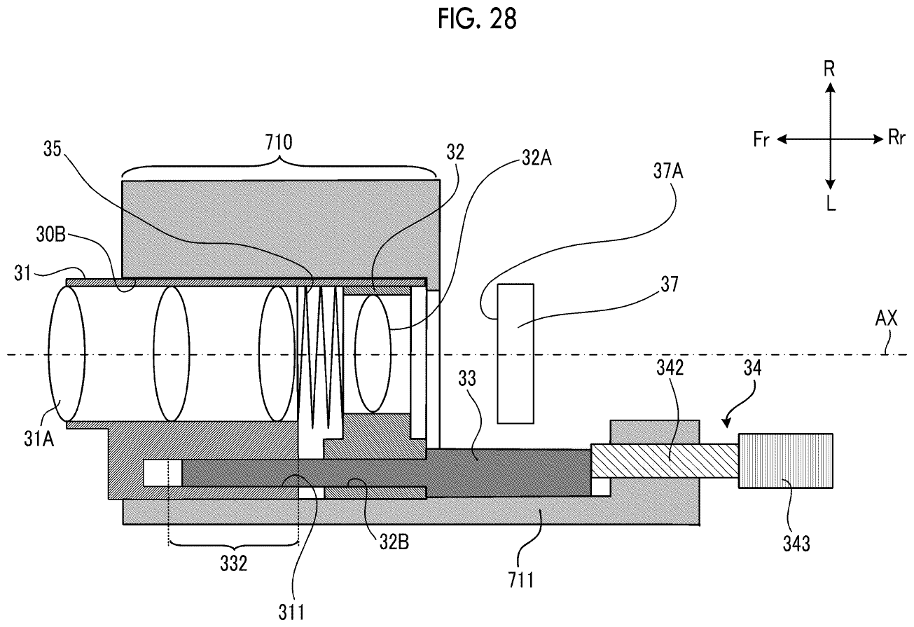

FIG. 28 is a schematic view showing a ninth modification example of the imaging unit 300 and the housing member 71, and is a view taken along line A-A shown in FIG. 6. The example shown in FIG. 28 is different from the example shown in FIG. 14 in that the fixed lens holder 31 is provided with the insertion hole 311 into which the distal end side of the shaft member 33 is inserted and the inner peripheral surface of the insertion hole 311 constitutes the second region 332 (first restriction part), the compression spring 35 is provided between the movable lens holder 32 and the fixed lens group, the base end side of the shaft member 33 relative to the movable lens holder 32 is not restricted in the up-down direction and the left-right direction by the extension portion 711, and the prism 36 is removed and the light-receiving surface 37A of the sensor 37 is disposed perpendicular to the optical axis AX. The actuator 34 shown in FIG. 28 can be changed to the sliding screw 340, the flexible shaft 341, and the motor. In the configuration shown in FIG. 28, the prism 36 may be added such that the light-receiving surface 37A of the sensor 37 is parallel to the optical axis AX. According to the configuration shown in FIG. 28, the lengths of the imaging unit 300 and the housing member 71 in the optical axis direction can be reduced.

In all the configurations described above, the relative position between the sensor 37 and the prism 36 (only the sensor 37 in a case in which the prism 36 is not necessary) and the housing member 71 is preferably determined as follows.

The relative position between the housing member 71 and the sensor 37 (and the prism 36) is determined by finely adjusting the sensor 37 (and the prism 36) in the directions perpendicular to the optical axis AX such that the optical axis AX is at the center of the effective pixel region of the sensor 37 and by finely adjusting the sensor 37 (and the prism 36) in the optical axis direction such that the best in-focus position is at the target position, and the sensor 37 (and the prism 36) is fixed to the housing member 71 with an adhesive at the determined position.

In the present specification, “center” means the substantial center, and a location near the center is interpreted as “center.”

As described above, at least the following matters are described in the present specification. In the following parentheses, the components and the like corresponding to those in the above-described embodiment are described, but the technology of the present disclosure is not limited to this.
(1)

An endoscope (endoscope 1) comprising: an insertion part (insertion part 2) that is inserted into a subject; an optical system (optical system OP) that is disposed on a distal end side of the insertion part and includes a first lens (movable lens 32A); a sensor (sensor 37) that captures an optical image of the subject formed by the optical system; a first holding part (movable lens holder 32) that holds the first lens; a shaft member (shaft member 33) that is fixed to the first holding part and extends toward a base end side relative to the first holding part; an actuator (actuator 34) that moves the shaft member in an optical axis direction along an optical axis of the optical system; and a first restriction part (first region 331, second region 332) that restricts a position of the shaft member in directions perpendicular to the optical axis, in which the directions perpendicular to the optical axis are a first direction (left-right direction) and a second direction (up-down direction) perpendicular to the first direction, a length (maximum length 33La, 33Lb) of the shaft member in the first direction and the second direction is shorter than a diameter (diameter φ) of the first lens, and a length (length LA, length L10) between a distal end of the first restriction part and a base end of the first restriction part in the optical axis direction is longer than a length (length LB) of the first lens in the optical axis direction.
(2)

The endoscope according to (1), further comprising: a housing member (housing member 71) having a first hole (insertion hole 30B) into which the optical system is inserted and a second hole (insertion hole 711A) into which the shaft member is inserted.
(3)

The endoscope according to (2), in which the first restriction part is provided on an inner periphery of the second hole.
(4)

The endoscope according to (2) or (3), in which the optical system includes a second lens (fixed lens group) and a second holding part (fixed lens holder 31) that holds the second lens, and the second holding part is fixed to the housing member.
(5)

The endoscope according to (1), in which the optical system includes a second lens (fixed lens group) and a second holding part (fixed lens holder 31) that holds the second lens, and the second holding part has a hole portion (insertion hole 311) that forms the first restriction part and into which the shaft member is inserted.
(6)

The endoscope according to (5), further comprising: a housing member (housing member 71) having a hole portion (insertion hole 30B) into which the second holding part is inserted.
(7)

The endoscope according to any one of (2) to (6), in which the housing member has a third hole (insertion hole 22B, insertion hole 24B) into which a light guide is inserted.
(8)

The endoscope according to any one of (1) to (7), in which a base end of the shaft member is located on a base end side relative to a light-receiving surface of the sensor.
(9)

The endoscope according to any one of (1) to (8), in which in a case in which the insertion part is viewed from a distal end side, the actuator and the sensor overlap each other.
(10)

The endoscope according to any one of (1) to (9), in which the optical system includes a prism (prism 36), and a base end of the shaft member is located on a base end side relative to a light incident surface (light incident surface 36A) of the prism.
(11)

The endoscope according to (10), in which the sensor is disposed such that a light-receiving surface is along the optical axis and the light-receiving surface faces a light emission surface of the prism.
(12)

The endoscope according to any one of (1) to (11), in which the first direction is a direction in which the first lens and the shaft member are arranged, and in the first direction, the shaft member is provided on a side opposite to the sensor with the optical axis interposed therebetween.
(13)

The endoscope according to any one of (1) to (12), in which the sensor is provided on a substrate (substrate 38), the substrate includes a first substrate portion (first substrate portion 381) in which the sensor is provided and a second substrate portion (second substrate portion 382) located on a base end side relative to the first substrate portion, and in a case of being viewed from a direction perpendicular to the light-receiving surface of the sensor, a center (center 382A) of the second substrate portion in a direction along a short side of the sensor is shifted, relative to a center (center 381A) of the first substrate portion in the direction along the short side of the sensor, in a direction away from the shaft member and the actuator.
(14)

The endoscope according to any one of (1) to (13), in which any functional element of an observation window (observation window 30), a first illumination window (first illumination window 24), a second illumination window (second illumination window 22), a forceps port (forceps port 26), or an air/water supply nozzle (air/water supply nozzle 27) is provided on a distal end surface (distal end surface 14) of the insertion part, and in a case in which the distal end surface is viewed in the optical axis direction, a center of the shaft member is located in a region interposed between a line extending from a center of the distal end surface and passing through a center of the observation window and a line extending from the center of the distal end surface and passing through a center of the functional element.
(15)

The endoscope according to (14), in which in a case in which the distal end surface is viewed in the optical axis direction, the center of the shaft member is located on an outer edge side of the distal end surface relative to the center of the observation window and the center of the functional element.
(16)

The endoscope according to (14), in which in a case in which the distal end surface is viewed in the optical axis direction, the center of the shaft member is located on a center side of the distal end surface relative to the center of the observation window and the center of the functional element.
(17)

The endoscope according to any one of (1) to (13), in which an observation window (observation window 30) and a plurality of functional elements are provided on a distal end surface (distal end surface 14) of the insertion part, in a case in which the distal end surface is viewed in the optical axis direction, a center of the shaft member is located on an outer edge side or a center side of the distal end surface relative to a center of the observation window and centers of the plurality of functional elements, and the plurality of functional elements are any of an illumination window, an observation window, a forceps port, or an air/water supply nozzle.
(18)

The endoscope according to (17), in which a maximum length (maximum length 33La, 33Lb) of the shaft member in the first direction and the second direction is shorter than a maximum length of the plurality of functional elements in the first direction and the second direction.
(19)

The endoscope according to any one of (1) to (18), in which the first lens includes a focus lens or a zoom lens.
(20)

The endoscope according to any one of (1) to (19), in which the actuator includes a piezoelectric element (piezoelectric element 343) and a movable part (rod 342) that is movable in the optical axis direction by an operation of the piezoelectric element, and moves the shaft member by movement of the movable part.
(21)

The endoscope according to (20), further comprising: a position detection unit (magnet MG, Hall element HA) provided on a base end side of the piezoelectric element.
(22)

The endoscope according to any one of (1) to (19), in which the actuator includes a motor and a movable part (sliding screw 340) that is movable in the optical axis direction by rotation of the motor, and moves the shaft member by movement of the movable part.
(23)

The endoscope according to (22), further comprising: a position detection unit (magnet MG, Hall element HA) provided on a base end side of the shaft member.
(24)

The endoscope according to any one of (1) to (23), in which a diameter of a distal end portion of the insertion part is 6 mm or less.
(25)

The endoscope according to any one of (1) to (24), in which a difference between a diameter of a distal end portion of the insertion part and a diameter of a forceps port is less than 6 mm.
(26)

The endoscope according to any one of (1) to (25), in which in the optical axis direction, the base end of the first restriction part is located on a base end side relative to a base end of a region (movement region R1) in which the first lens is movable.
(27)

The endoscope according to any one of (1) to (26), in which in the optical axis direction, the distal end of the first restriction part is located on a distal end side relative to a distal end of a region (movement region R1) in which the first lens is movable.
(28)

The endoscope according to any one of (1) to (27), in which the first restriction part has a first region (first region 331) located on a base end side relative to a base end of a region (movement region R1) in which the first lens is movable in the optical axis direction, and a second region (second region 332) located on a distal end side relative to a distal end of the region (movement region R1) in which the first lens is movable in the optical axis direction, and a length (length L10) from a base end of the first region to a distal end of the second region is longer than the length (length LB) of the first lens in the optical axis direction.
(29)

The endoscope according to any one of (1) to (28), in which the shaft member has a circular cross section, and the endoscope further comprises: a second restriction part (second restriction part 322) that restricts rotation of the first holding part around an axis of the shaft member.
(30)

The endoscope according to (29), in which the second restriction part is provided around a region (region 320) of the first holding part that houses the first lens.
(31)

The endoscope according to any one of (1) to (30), in which an end surface (end surface 33Rr) of the shaft member on a base end side and an end surface (end surface 340Fr) of the actuator on a distal end side are in contact with each other.
(32)

The endoscope according to any one of (1) to (31), in which the first direction is a direction in which the first lens and the shaft member are arranged, and in the first direction, a distance (distance L6) between a centroid (centroid 34C) of the actuator and the optical axis is shorter than a distance (distance L7) between a centroid (centroid 33C) of the shaft member and the optical axis.
(33)

The endoscope according to any one of (1) to (32), in which the first direction is a direction in which the first lens and the shaft member are arranged, in the first direction, one end (one end E1), on an optical system side, of an end surface (end surface 33Rr) of the shaft member on a base end side is located on a side opposite to the optical system side relative to one end (one end E2), on the optical system side, of an end surface (end surface 340Fr) of the actuator on a distal end side, and the other end (the other end E3), on a side opposite to the optical system side, of the end surface of the shaft member on the base end side is located on a side opposite to the optical system side relative to the other end (the other end E4), on a side opposite to the optical system side, of the end surface of the actuator on the distal end side.
(34)

The endoscope according to any one of (1) to (33), further comprising: a biasing member (compression spring 35, magnet MG) that biases the first holding part in the optical axis direction, in which in a case in which a position of the first lens in the optical axis direction is a first position (position other than a base end of a movement region R1 in a front-rear direction), an end surface of the shaft member on a base end side and an end surface of the actuator on a distal end side are in contact with each other, and in a case in which the position of the first lens in the optical axis direction is a second position (position of a base end of a movement region R1 in a front-rear direction) different from the first position, the end surface of the shaft member on the base end side and the end surface of the actuator on the distal end side are not in contact with each other.
(35)

The endoscope according to (34), in which the actuator is provided on a base end side relative to the first holding part, and the biasing member is provided on a distal end side relative to the first holding part to bias the first holding part toward a base end side.

### Explanation of References

1: endoscope
2: insertion part
3: operating part
4: universal cord
5: soft portion
6: bendable portion
7: distal end portion
8, 9: angle knob
10: air/water supply button
12: treatment tool inlet port
14: distal end surface
22: second illumination window
22A, 22B, 24A, 24B, 26A, 26B, 27A: insertion hole
30A, 30B, 710A, 711A: insertion hole
24: first illumination window
26: forceps port
27: air/water supply nozzle
30: observation window
31: fixed lens holder
31A: objective lens
32: movable lens holder
32A: movable lens
32B: through-hole
32L, 32R, 320, 321, 323: region
32X: engaging protrusion
33: shaft member
33C, 34C: centroid
33La, 33Lb: length
33Rr, 340Fr, 342Fr: end surface
34: actuator
35: compression spring
36: prism
36A: light incident surface
37: sensor
37A: light-receiving surface
38: substrate
39: cable
62: connecting member
71, 71A: housing member
72: distal end cap
91: first housing region
92: second housing region
220: left light guide
240: right light guide
260: treatment tool pipe line
270: fluid pipe line
300: imaging unit
310, 322A, 322D: protrusion
311: insertion hole
322: second restriction part
322B: member
322C: leaf spring
322E: recessed portion
331: first region
332: second region
340: sliding screw
341: flexible shaft
342: rod
343: piezoelectric element
381: first substrate portion
381A, 382A, CP: center
382: second substrate portion
710: body portion
710S: inner peripheral surface
711, 712: extension portion
E1, E2: one end
E3, E4: other end
L1, L4, L5, L6, L7: distance
P1: first portion
P2: second portion
P3: third portion

What is claimed is:

1. An endoscope comprising:
an insertion part that is inserted into a subject;
an optical system that is disposed on a distal end side of the insertion part and includes a first lens;
a sensor that captures an optical image of the subject formed by the optical system;
a first holding part that holds the first lens;
a shaft member that is fixed to the first holding part and extends toward a base end side relative to the first holding part;
an actuator that moves the shaft member in an optical axis direction along an optical axis of the optical system; and
a first restriction part that restricts a position of the shaft member in directions perpendicular to the optical axis,
wherein the directions perpendicular to the optical axis include a first direction and a second direction perpendicular to the first direction,
a length of the shaft member in the first direction and a length of the shaft member in the second direction are shorter than a diameter of the first lens, and
a length between a distal end of the first restriction part and a base end of the first restriction part in the optical axis direction is longer than a length of the first lens in the optical axis direction.

2. The endoscope according to claim 1, further comprising:
a housing member having a first hole into which the optical system is inserted and a second hole into which the shaft member is inserted.

3. The endoscope according to claim 2,
wherein the first restriction part is provided on an inner periphery of the second hole.

4. The endoscope according to claim 2,
wherein the optical system includes a second lens and a second holding part that holds the second lens, and
the second holding part is fixed to the housing member.

5. The endoscope according to claim 1,
wherein the optical system includes a second lens and a second holding part that holds the second lens, and
the second holding part has a hole portion that forms the first restriction part and into which the shaft member is inserted.

6. The endoscope according to claim 5, further comprising:
a housing member having a hole portion into which the second holding part is inserted.

7. The endoscope according to claim 2,
wherein the housing member has a third hole into which a light guide is inserted.

8. The endoscope according to claim 1,
wherein a base end of the shaft member is located on a base end side relative to a light-receiving surface of the sensor.

9. The endoscope according to claim 8,
wherein when the insertion part is viewed from a distal end side, the actuator and the sensor overlap each other.

10. The endoscope according to claim 1,
wherein the optical system includes a prism, and
a base end of the shaft member is located on a base end side relative to a light incident surface of the prism.

11. The endoscope according to claim 10,
wherein the sensor is disposed such that a light-receiving surface of the sensor is along the optical axis and the light-receiving surface faces a light emission surface of the prism.

12. The endoscope according to claim 11,
wherein the first direction is a direction in which the first lens and the shaft member are arranged, and
in the first direction, the shaft member is provided on a side opposite to the sensor with the optical axis interposed therebetween.

13. The endoscope according to claim 11,
wherein the sensor is provided on a substrate,
the substrate includes a first substrate portion in which the sensor is provided and a second substrate portion located on a base end side relative to the first substrate portion, and
when viewed from a direction perpendicular to the light-receiving surface of the sensor, a center of the second substrate portion in a direction along a short side of the sensor is shifted, relative to a center of the first substrate portion in the direction along the short side of the sensor, in a direction away from the shaft member and the actuator.

14. The endoscope according to claim 1,
wherein any functional element of an observation window, a first illumination window, a second illumination window, a forceps port, or an air/water supply nozzle is provided on a distal end surface of the insertion part, and
when the distal end surface is viewed in the optical axis direction, a center of the shaft member is located in a region interposed between a line extending from a center of the distal end surface and passing through a center of the observation window and a line extending from the center of the distal end surface and passing through a center of the functional element.

15. The endoscope according to claim 14,
wherein when the distal end surface is viewed in the optical axis direction, the center of the shaft member is located on an outer edge side of the distal end surface relative to the center of the observation window and the center of the functional element.

16. The endoscope according to claim 14,
wherein when the distal end surface is viewed in the optical axis direction, the center of the shaft member is located on a center side of the distal end surface relative to the center of the observation window and the center of the functional element.

17. The endoscope according to claim 1,
wherein an observation window and a plurality of functional elements are provided on a distal end surface of the insertion part,
when the distal end surface is viewed in the optical axis direction, a center of the shaft member is located on an outer edge side or a center side of the distal end surface relative to a center of the observation window and centers of the plurality of functional elements, and
the plurality of functional elements are any of an illumination window, an observation window, a forceps port, or an air/water supply nozzle.

18. The endoscope according to claim 17,
wherein a maximum length of the shaft member in the first direction and the second direction is shorter than a maximum length of the plurality of functional elements in the first direction and the second direction.

19. The endoscope according to claim 1,
wherein the actuator includes a piezoelectric element and a movable part that is movable in the optical axis direction by an operation of the piezoelectric element, and moves the shaft member by movement of the movable part.

20. The endoscope according to claim 19, further comprising:
a position detection unit provided on a base end side of the piezoelectric element.

21. The endoscope according to claim 1,
wherein a diameter of a distal end portion of the insertion part is 6 mm or less.

22. The endoscope according to claim 1,
wherein a difference between a diameter of a distal end portion of the insertion part and a diameter of a forceps port is less than 6 mm.

23. The endoscope according to claim 1,
wherein in the optical axis direction, the base end of the first restriction part is located on a base end side relative to a base end of a region in which the first lens is movable.

24. The endoscope according to claim 1,
wherein in the optical axis direction, the distal end of the first restriction part is located on a distal end side relative to a distal end of a region in which the first lens is movable.

25. The endoscope according to claim 1,
wherein the first restriction part has a first region located on a base end side relative to a base end of a region in which the first lens is movable in the optical axis direction, and a second region located on a distal end side relative to a distal end of the region in which the first lens is movable in the optical axis direction, and
a length from a base end of the first region to a distal end of the second region is longer than the length of the first lens in the optical axis direction.

26. The endoscope according to claim 1,
wherein an end surface of the shaft member on a base end side and an end surface of the actuator on a distal end side are in contact with each other.

27. The endoscope according to claim 1,
wherein the first direction is a direction in which the first lens and the shaft member are arranged, and
in the first direction, a distance between a centroid of the actuator and the optical axis is shorter than a distance between a centroid of the shaft member and the optical axis.

28. The endoscope according to claim 1,
wherein the first direction is a direction in which the first lens and the shaft member are arranged,
in the first direction, one end, on an optical system side, of an end surface of the shaft member on a base end side is located on a side opposite to the optical system side relative to one end, on the optical system side, of an end surface of the actuator on a distal end side, and
the other end, on a side opposite to the optical system side, of the end surface of the shaft member on the base end side is located on a side opposite to the optical system side relative to the other end, on a side opposite to the optical system side, of the end surface of the actuator on the distal end side.

29. The endoscope according to claim 1, further comprising:
a biasing member that biases the first holding part in the optical axis direction,
wherein in a case in which a position of the first lens in the optical axis direction is a first position, an end surface of the shaft member on a base end side and an end surface of the actuator on a distal end side are in contact with each other, and
in a case in which the position of the first lens in the optical axis direction is a second position different from the first position, the end surface of the shaft member on the base end side and the end surface of the actuator on the distal end side are not in contact with each other.

30. The endoscope according to claim 29,
wherein the actuator is provided on a base end side relative to the first holding part, and
the biasing member is provided on a distal end side relative to the first holding part to bias the first holding part toward a base end side.
